(19) United States
(12) Patent Application Publication
(10) Pub. No.: US 2025/0360171 A1
(43) Pub. Date: Nov. 27, 2025

(54) CHEMOGENETICALLY GATED ION CHANNELS AND USE THEREOF

(71) Applicants: TECHNION RESEARCH & DEVELOPMENT FOUNDATION LIMITED, Haifa (IL); RAMBAM MED-TECH LTD., Haifa (IL)

(72) Inventors: Lior GEPSTEIN, Haifa (IL); Yehuda WEXLER, Haifa (IL)

(21) Appl. No.: 19/276,180

(22) Filed: Jul. 22, 2025

Related U.S. Application Data
(63) Continuation of application No. PCT/IL2024/050090, filed on Jan. 22, 2024.
(60) Provisional application No. 63/440,421, filed on Jan. 22, 2023.

(51) Int. Cl. A61K 35/34; A61K 38/17; C07K 14/705; C12N 15/90

(52) U.S. Cl. CPC A61K 35/34; A61K 38/177; C07K 14/705; C12N 15/907; C07K 2319/00; C12N 2800/90; C12N 2830/50

(57) ABSTRACT

Nucleic acid molecules comprising a first sequence encoding a cyclic nucleotide gated potassium channel or a functional fragment thereof and a second sequence encoding a chemogenetically activatable cyclic adenosine monophosphate (cAMP) generating receptor or a functional fragment thereof are provided. Expression vectors comprising the molecules, fusion proteins encoded by the molecules, cells, kits and pharmaceutical compositions are also provided. Methods of hyperpolarizing a cell, depolarizing a cardiac cell and treating a disease or condition are also provided.

### CROSS REFERENCE TO RELATED APPLICATIONS

This application is a ByPass Continuation of PCT Patent Application No. PCT/IL2024/050090 having International filing date of Jan. 22, 2024, which claims the benefit of priority of U.S. Provisional Patent Application No. 63/440,421, filed Jan. 22, 2023, the contents of which are all incorporated herein by reference in their entirety.

### REFERENCE TO AN ELECTRONIC SEQUENCE LISTING

The contents of the electronic sequence listing (TECH-TRMB-P-0281-PCT.xml; Size: 46,012 bytes; and Date of Creation: Jan. 11, 2024) is herein incorporated by reference in its entirety.

### FIELD OF INVENTION

The present invention is in the field of cardiac therapy and chemogenetic modulation.

### BACKGROUND OF THE INVENTION

Anti-arrhythmic pharmacotherapies have been hampered by their global cardiac action, low efficacy, and significant pro-arrhythmic effects. Additionally, myocardial cell therapy procedures using cardiomyocytes have been hindered by the inability to control the transplanted cell's excitable properties, especially when displaying arrhythmic activity. Hence, a method allowing targeted, externally-controlled, electrophysiological modulation of native myocardium or transplanted cells is highly desirable.

### SUMMARY OF THE INVENTION

The present invention provides nucleic acid molecules comprising a first sequence encoding a cyclic nucleotide gated potassium channel or a functional fragment thereof and a second sequence encoding a chemogenetically activatable cyclic adenosine monophosphate (cAMP) generating receptor or a functional fragment thereof. Expression vectors comprising the molecules, fusion proteins encoded by the molecules, cells, kits and pharmaceutical compositions are also provided. Methods of hyperpolarizing a cell, depolarizing a cardiac cell and treating a disease or condition are also provided.

According to a first aspect, there is provided a nucleic acid molecule comprising a first sequence encoding a cyclic nucleotide gated potassium channel or a functional fragment thereof and a second sequence encoding a chemogenetically activatable cyclic nucleotide generating receptor or a functional fragment thereof.

According to some embodiments, the cyclic nucleotide gated potassium channel is SthK.

According to some embodiments, a sequence encoding SthK comprises SEQ ID NO: 1 or a sequence with at least 85% identity thereto.

According to some embodiments, the chemogenetically activatable cyclic nucleotide generating receptor is a receptor activatable by a synthetic ligand.

According to some embodiments, the chemogenetically activatable cyclic nucleotide generating receptor is a DREADD.

According to some embodiments, the DREADD is an excitatory DREADD.

According to some embodiments, the DREADD is derived from the human M3 muscarinic receptor (hM3).

According to some embodiments, the DREADD is rM3D.

According to some embodiments, a sequence encoding rM3D comprises SEQ ID NO: 2 or a sequence with at least 85% identity thereto.

According to some embodiments, the first sequence and the second sequence are in the same open reading frame.

According to some embodiments, the nucleic acid molecule is a DNA molecule or an RNA molecule.

According to some embodiments, the nucleic acid molecule is a DNA molecule and wherein a single open reading frame encodes an mRNA translatable to the cyclic nucleotide gated potassium channel and the chemogenetically activatable cAMP generating receptor.

According to some embodiments, the nucleic acid molecule comprises a third sequence encoding a linker peptide between the first sequence and the second sequence.

According to some embodiments, the linker peptide is a cleavable peptide.

According to some embodiments, the linker peptide is a P2A peptide.

According to some embodiments, a sequence encoding a P2A peptide comprises SEQ ID NO: 3.

According to some embodiments, the nucleic acid molecule encodes a protein comprising or consisting of SEQ ID NO: 8.

According to some embodiments, the nucleic acid molecule comprises SEQ ID NO: 4.

According to another aspect, there is provided an expression vector comprising a nucleic acid molecule of the invention operatively linked to at least one transcriptional regulatory element.

According to some embodiments, the at least one transcriptional regulatory element is a promoter.

According to some embodiments, the promoter is a constitutive promoter or a promoter specifically active in cardiac cells.

According to some embodiments, the at least one transcriptional regulatory element comprises at least cardiac cell specific enhancer.

According to some embodiments, there is provided a fusion protein comprising a cyclic nucleotide gated potassium channel or a functional fragment thereof and a chemogenetically activatable cAMP generating receptor or a functional fragment thereof.

According to some embodiments, the cyclic nucleotide gated potassium channel is SthK, the chemogenetically activatable cAMP generating receptor is rM3D or both.

According to some embodiments, the SthK comprises SEQ ID NO: 5 or a functional fragment thereof or sequence with at least 85% identity thereto, the rM3D comprises SEQ ID NO: 6 or a functional fragment thereof or sequence with at least 85% identity thereto, or both.

According to some embodiments, the fusion protein is encoded by a nucleic acid molecule of the invention.

According to some embodiments, the fusion protein comprises the amino acid sequence provided in SEQ ID NO: 28.

According to another aspect, there is provided a cell comprising a nucleic acid molecule of the invention, an expression vector or the invention or a fusion protein of the invention.

According to some embodiments, the cell is a cardiac cell, optionally wherein the cell is a cardiomyocyte.

According to another aspect, there is provided a pharmaceutical composition comprising a nucleic acid molecule of the invention, an expression vector of the invention, a fusion protein of the invention or a cell of the invention and a pharmaceutically acceptable carrier, excipient or adjuvant.

According to some embodiments, the pharmaceutical composition is formulated for administration to a subject.

According to another aspect, there is provided a method of hyperpolarizing a cell, the method comprising expressing in the cell a nucleic acid molecule of the invention, an expression vector of the invention or a fusion protein of the invention and contacting the cell with a ligand of the chemogenetically activatable cyclic nucleotide generating receptor, thereby hyperpolarizing a cell.

According to some embodiments, the ligand is clozapine-N-oxide (CNO) or DREADD agonist 21/compound 21 (C21).

According to some embodiments, the cell is a cardiac cell, optionally wherein the cell is a cardiomyocyte.

According to another aspect, there is provided a method of treating or preventing a disease or condition in a subject in need thereof, the method comprising administering to the subject a pharmaceutical composition of the invention and further administering a ligand of the chemogenetically activatable cyclic nucleotide generating receptor, thereby treating a cardiac disease or condition.

According to some embodiments, the ligand is CNO or C21.

According to some embodiments, the disease or condition is characterized by electrical disfunction in a disease tissue or cell.

According to some embodiments, the disease or condition is a cardiac disease or condition.

According to some embodiments, the cardiac disease or condition is selected from: arrhythmia, tachy-arrhythmia, brady-arrhythmia, bradycardia and tachycardia.

According to some embodiments, the disease or condition is a neurological disease or condition caused by hyperactivity of a neuron.

According to some embodiments, the neurological disease or condition is selected from: epilepsy, Parkinson's and parkinsonian syndromes, essential tremor, restless leg syndrome, tinnitus, pain, and phantom sensations, Alzheimer's disease and neuropathy.

According to some embodiments, the disease or condition is a smooth muscle disease or condition.

According to some embodiments, the smooth muscle disease or condition is selected from benign prostatic hyperplasia (BPH), hypertension, erectile dysfunction, coronary artery disease, pathologies of the stomach and intestines leading to lack of motility or hyper motility, achalasia, gastroesophageal reflux disease (GERD), urinary incontinence and urinary retention.

According to some embodiments, the disease or condition is a striated muscle disease or condition requiring muscle relaxation.

According to another aspect there is provided a nucleic acid molecule of the invention, an expression vector of the invention, a fusion protein of the invention, a cell of the invention or a pharmaceutical composition of the invention for use in treating a disease or condition characterized by electrical disfunction in a disease tissue or cell in a subject in need thereof.

A kit comprising:
a. a nucleic acid molecule of the invention, an expression vector of the invention, a fusion protein of the invention, a cell of the invention or a pharmaceutical composition of the invention; and
b. a ligand of the chemogenetically activatable cyclic nucleotide generating receptor.

According to some embodiments, the ligand is CNO or C21.

According to another aspect, there is provided a method of depolarizing a cardiac cell, the method comprising expressing in the cardiac cell a PSAM4-5HT3 fusion protein and contacting the cardiac cell with a ligand of PSAM4, thereby depolarizing a cardiac cell.

According to another aspect, there is provided a pharmaceutical composition comprising a cardiac cell expressing a PSAM4-5HT3 fusion protein and a pharmaceutically acceptable carrier, excipient or adjuvant.

According to another aspect, there is provided a method of treating or preventing a cardiac disease or condition in a subject in need thereof the method comprising administering to the subject a PSAM4-5HT3 fusion protein, a nucleic acid molecule encoding the PSAM4-5HT3 fusion protein or a pharmaceutical composition of the invention, and administering to the subject a ligand of PSAM4, thereby treating a cardiac disease or condition.

According to some embodiments, the ligand is Varenicline.

According to some embodiments, a low dose of the ligand causes increased electrical activity of the cardiac cell or adjacent cardiac cells and a high dose of the ligand causes complete silencing of electrical activity in the cardiac cell or adjacent cardiac cells.

According to some embodiments, a low dose is a subclinical dose and a high dose is at least a clinical dose.

According to some embodiments, a clinical dose is 0.5 mg of Varenicline.

According to some embodiments, the disease or condition is selected from arrythmia, tachy-arrhythmia, brady-arrhythmia, bradycardia and tachycardia.

According to some embodiments, the PSAM4-5HT3 comprises SEQ ID NO: 12.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Creation of the stable PSAM4-5HT3-hiPSC line. Inserted genetic construct (top); overlay of phase-contrast and GFP images of PSAM4-5HT3-hiPSCs (scale-bar: 100 um, bottom-left) showing cells are GFP positive, and immunostaining of PSAM4-5HT3-hiPSC-CMs (bottom-right) for cTnT (red), alpha-actinin (orange), and GFP (green).

FIGS. 2A-2D: Cellular electrophysiological characterization of PSAM4-5HT3-hiPSC-CMs. (2A) Representative current and (2B) voltage measurements over time following 10 nM Varenicline. Varenicline led to significant membrane depolarization, generation of a single low-amplitude AP (arrow), followed by suppression of automaticity. (2C) Comparison of mean normalized-current and maximum membrane resting-potential following Varenicline administration in PSAM4-5HT3-hiPSC-CMs and non-transfected isogenic-controls. (2D) Picrotoxin (100 uM) partially reversed Varenicline-induced depolarizing current. Mean±SEM, paired t-test. *p<0.05; **p<0.01; ****p<0.0001; ns: p>0.05. One-way ANOVA with multiple comparisons using Tukey's correction.

FIGS. 3A-3D: Tissue electrophysiological characterization. (3A) Phase-contrast and GFP images of a PSAM4-5HT3-hiPSC-CCS (Scale-bar: 1 mm). CCS is GFP positive throughout. (3B-3D) (3B) Normalized dose-response-curve, (3C) representative optical-signals, and (3D) summarizing bar graph depicting Varenicline-induced changes in spontaneous beating rates of PSAM4-5HT3-hiPSC-CCSs. *p<0.05; **p<0.01; ****p<0.0001; ns: p>0.05. One-way ANOVA with multiple comparisons using Tukey's correction.

FIGS. 4A-4D: Co-culture model. (4A) Fluorescent image showing the PSAM4-5HT3 (cGFP, green) and non-transfected CMs (left) areas within the co-culture. (4B-C) Co-culture activation-maps demonstrating (4B) slow-spontaneous activity arising from control cells (red, upper left corner) at baseline and (4C) a fast (>6-fold increase) Varenicline-induced rhythm arising from the PSAM4-5HT3-hiPSC-CMs (bottom right corner). (4D) Bar-graph summarizing the spontaneous activation rates following escalating Varenicline doses. Grey/black/white denote activities originating from the PSAM4-5HT3, control, and interphase co-culture regions respectively. *p<0.05; ** p<0.01; **** p<0.0001; ns: p>0.05. One-way ANOVA with multiple comparisons using Tukey's correction.

FIGS. 5A-5B: Chemogenetic silencing of electrical activity. Optical-recordings (right-panels) and activation maps (left-panels) of the co-cultures (5A) at baseline and (5B) following high-concentration (100 nM) Varenicline, which silenced electrical activity in PSAM4-5HT3-hiPSC-CMs area.

FIGS. 6A-6B: Chemogenetics for biological pacemaking in-vivo. Optical mapping of Langendorff-perfused (6A) chemogenetic-hiPSC-CMs engrafted and (6B) control (non-transplanted) rat hearts. Shown are optical maps during baseline recordings (left-panels) and following application of 30 nM Varenicline (“VAR”) and adenosine (“ADN”) (right-panels). Notice that electrical activity originated (red) from the septum in the control heart (both conditions) and at baseline in the treated heart. During Varenicline and adenosine application, a ventricular ectopic rhythm emerged (red) from the area of cell engraftment (lateral-apex).

FIG. 7A-7C: Optical traces in cardiac cell sheets expressing SthK and Rm3D (7A) after induction of arrythmia, (7B) upon termination of arrythmia by addition of CNO and (7C) after CNO washout showing normal rhythm.

FIG. 8: Diagram of the layout of two wild-type CCSs separated by a CCS of cardiomyocytes expressing the compound channel of the invention.

FIG. 9: Optical trace from cardiac cell sheet in which 10% of cells are engineered to express SthK and Rm3D and 90% of cells are control non-engineered cardiomyocytes showing termination of arrythmia.

### DETAILED DESCRIPTION OF THE INVENTION

The present invention provides nucleic acid molecules comprising a first sequence encoding a cyclic nucleotide gated potassium channel or a functional fragment thereof and a second sequence encoding a chemogenetically activatable cyclic adenosine monophosphate (cAMP) generating receptor or a functional fragment thereof. Expression vectors comprising the molecules are provided. Fusion proteins encoded by the molecules are provided. Cells comprising the molecules or fusion proteins are provided. Kits comprising the molecules or fusion proteins are provided. Pharmaceutical compositions comprising the molecules, fusion proteins or cells are also provided. Methods of hyperpolarizing a cell and/or treating a disease or condition are provided. Methods of depolarizing a cardiac cell and/or treating a cardiac disease or condition by expressing a PSAM4-5HT3 fusion protein in a cardiac cell are also provided.

Gene and cell therapies hold great therapeutic promise for cardiac arrhythmias and heart failure. The invention is based, at least in part, on the surprising finding that expression of a chemogenetic ion-channel (fusion protein) permits precise, localized and reversible functional perturbations of the cardiac tissue's electrophysiological properties using sub-therapeutic doses of an FDA-approved drug. Given the bidirectional effects achieved, increasing automaticity in a dose-dependent manner at low Varenicline concentrations and suppressing automaticity and excitability at higher doses, this chemogenetic strategy can be utilized in a wide spectrum of applications. The ability to reversibly silence electrical activity only at a desired location brings unique value to the treatment of tachyarrhythmias, overcoming the limitations of traditional anti-arrhythmic drugs. Finally, this technology can improve the safety of myocardial cell therapy, as episodes of ventricular tachyarrhythmias have previously been noted following CM engraftment in large animals (Liu et al., “Human embryonic stem cell-derived cardiomyocytes restore function in infarcted hearts of non-human primates”, Nature Biotechnology, 2018;36:597-605). By using chemogenetics to silence the activity of the transplanted cells, one can introduce a safety mechanism for prevention/termination of arrhythmias.

By a first aspect, there is provided a nucleic acid molecule comprising a first sequence encoding a cyclic nucleotide gated potassium channel or a fragment thereof and a second sequence encoding a chemogenetically activatable cyclic adenosine monophosphate (cAMP) generating receptor or a fragment thereof.

The term “nucleic acid” is well known in the art. “A nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C).

The terms “nucleic acid molecule” include but not limited to single-stranded RNA (ssRNA), double-stranded RNA (dsRNA), single-stranded DNA (ssDNA), double-stranded DNA (dsDNA), small RNA such as miRNA, siRNA and other short interfering nucleic acids, snoRNAs, snRNAs, tRNA, piRNA, tnRNA, small rRNA, hnRNA, IncRNA, circulating nucleic acids, fragments of genomic DNA or RNA, degraded nucleic acids, ribozymes, viral RNA or DNA, nucleic acids of infectious origin, amplification products, modified nucleic acids, plasmidical or organellar nucleic acids and artificial nucleic acids such as oligonucleotides. In some embodiments, the nucleic acid molecule is a DNA molecule. In some embodiments, the DNA molecule is a single stranded DNA molecule. In some embodiments, the DNA molecule is a double stranded DNA molecule. In some embodiments, the nucleic acid molecule is an RNA molecule.

In some embodiments, the first sequence is an open reading frame. In some embodiments, the first sequence is in a first open reading frame. In some embodiments, the second sequence is an open reading frame. In some embodiments, the second sequence is in a second open reading frame. In some embodiments, the first sequence and the second sequence are in the same open reading frame. In some embodiments, the first open reading frame and the second open reading frame are the same open reading frame. In some embodiments, the first open reading frame and the second open reading frame are different open reading frames. In some embodiments, the nucleic acid molecule is a DNA molecule and the first open reading frame encodes an mRNA translatable to the cyclic nucleotide gated potassium channel. In some embodiments, the nucleic acid molecule is a DNA molecule and the second open reading frame encodes an mRNA translatable to the chemogenetically activatable cAMP generating receptor. In some embodiments, the nucleic acid molecule is a DNA molecule and a single open reading frame encodes an mRNA translatable to the cyclic nucleotide gated potassium channel and the chemogenetically activatable cAMP generating receptor. In some embodiments, the first sequence is 5′ to the second sequence. In some embodiments, the second sequence is 5′ to the first sequence. In some embodiments, the first sequence is 3′ to the second sequence. In some embodiments, the second sequence is 3′ to the first sequence.

As used herein, the term “cyclic nucleotide” refers to a single-phosphate nucleotide with a cyclic bond arrangement between the sugar and phosphate group. In some embodiments, the cyclic nucleotide is a ribose nucleotide. In some embodiments, the cyclic nucleotide is a deoxyribose nucleotide. In some embodiments, the cyclic nucleotide comprises a bond between a phosphate group and 3′ hydroxyl group of the sugar. In some embodiments, the cyclic nucleotide comprises a bond between a phosphate group and 5′ hydroxyl group of the sugar. In some embodiments, the cyclic nucleotide comprises a first bond between a phosphate group and a 3′ hydroxyl group of the sugar and a second bond between the phosphate group and a 5′ hydroxyl group of the sugar. In some embodiments, the cyclic nucleotide is a cyclic monophosphate. In some embodiments, the cyclic nucleotide is a cyclic adenosine monophosphate (cAMP). In some embodiments, the cyclic nucleotide is a cyclic guanosine monophosphate (cGMP). In some embodiments, the cyclic nucleotide is a cyclic cytosine monophosphate (cCMP). In some embodiments, the cyclic nucleotide is a cyclic uridine monophosphate (cUMP). In some embodiments, the cyclic nucleotide is a cyclic thymidine monophosphate (cTMP). In some embodiments, the cyclic nucleotide is selected from cAMP and cGMP.

As used herein, the term “cyclic nucleotide gated channel” refers to an ion channel which is activated by the binding of a cyclic nucleotide. In some embodiments, activation of the channel comprises opening the channel. In some embodiments, the channel is a cAMP gated channel. In some embodiments, the channel is gated by cAMP and not by cGMP. In some embodiments, the channel is a potassium channel. In some embodiments, a potassium channel is a potassium selective pore that spans a cellular membrane. In some embodiments, the membrane is the plasma membrane. In some embodiments, the channel is specific to potassium. In some embodiments, the channel transports potassium into the cell. In some embodiments, the channel transports potassium out of the cell. In some embodiments, the channel transports positively charged ions. In some embodiments, the channel is not specific to potassium. In some embodiments, the channel transports only potassium. In some embodiments, the channel transports potassium and sodium. In some embodiments, the channel transports potassium and sodium but is preferential to potassium.

Gated channels and specifically gated potassium channels and specifically cyclic nucleotide gated channels are well known in the art and any such channel may be used. In some embodiments, the cyclic nucleotide gated potassium channel is from the CNG subfamily of channels. In some embodiments, the channel is a cyclic nucleotide-regulated channel. In some embodiments, the cyclic nucleotide gated potassium channel is from the hyperpolarization-activated and cyclic nucleotide-gated (HCN) subfamily of channels. In some embodiments, the cyclic nucleotide gated potassium channel is prokaryotic channel. Examples of prokaryotic cyclic nucleotide gated ion channels can be found in, for example, Brams et al., “Family of prokaryote cyclic nucleotide-modulated ion channels”, PNAS, 2014 May 27;111 (21): 7855-60, herein incorporated by reference in its entirety. In some embodiments, the prokaryotic channel is from E. coli. In some embodiments, the cyclic nucleotide gated potassium channel is SthK. SthK was selected both for its small size (allowing for easier transfer of the genetic material encoding it) and for its high conductance specifically of potassium. In contrast to other channels that require mutations to produce high conductance (see below the 5HT3-HC variant) the SthK channel naturally has very high conductance of potassium.

In some embodiments, SthK is a prokaryotic protein. In some embodiments, SthK is E. coli SthK. In some embodiments, the sequence encoding SthK comprises atgaaaagctccgccttctcccaccccacctacaccctggtctggaaagtcggcattctggctgtcactctgtattacgctattcgaat cccactgaccctggtgttcccctctctgtttagtcccctgctgcctctggatatcctggccagtctggctctgategcagacattcctct ggatttcgcctttgagtcacgaaagacaagcggcaggaaaccaactctgctggctcctagccgactgccagatctgctggccgct ctgccactggacctgctggtgttcgccctgcacctgccatcacccctgagcctgctgtccctggtgaggctgctgaagctgatctcc gtccagaggtctgctacaagaatcctgtcttacagaattaacccagcactgctgcggctgctgagtctggtgggattcatcctgctgg cagcccatgggattgcctgcggctggatgtcactgcagccacctagcgagtccccagcaggaaccagatacctgagegccttcta ctggacaatcaccacactgactaccateggctacggagatattaccccatccacacccattcagaccgtgtacaccatcgtcattga gctgctgggagctgcaatgtatggactggtcatcgggaatattgcatctctggtcagtaagctggacgccgctaaactgctgcacc gagagaggatggaacgggtgacagctttcctgagttacaagaaaatctcacctgagctgcagaggagaattctggaatactttgatt atctgtgggagactcggcgcgggtatgaggaacgcgaggtgctgaaggaactgcctcacccactgcgactggctgtcgcaatgg aaatccatggcgacgtgattgagaaggtcccactgttcaaaggggccggcgaagactttatccgcgatatcattctgcatctggag cccgtgatctacggacctggggaatatatcattagggctggcgagctgggcagcgatgtctactttatcaacagaggcagcgtgga ggtcctgtccgcagacgaaaagacccggtatgccatcctgtctgagggccagttctttggagaaatggcactgattctgcgagcac cacgaacagctactgtgagagcacggactttctgtgacctgtacagactggataaagaaacctttgacagaatcctgtctegctatc ctgagattgcagcccagattcaggaactggctgtgcggaggaaagaagaactggaaggggggacatcacggggggaaccgg tcccgggcttaaggagctcgcatgeggaagcgga (SEQ ID NO: 1). In some embodiments, the sequence encoding SthK consists of SEQ ID NO: 1. In some embodiments, the SthK protein comprises the amino acid sequence MKSSAFSHPTYTLVWKVGILAVTLYYAIRIPLTLVFPSLFSPLLPLDILASLALIADIP LDFAFESRKTSGRKPTLLAPSRLPDLLAALPLDLLVFALHLPSPLSLLSLVRLLKLIS VQRSATRILSYRINPALLRLLSLVGFILLAAHGIACGWMSLOPPSESPAGTRYLSAF YWTITTLTTIGYGDITPSTPIQTVYTIVIELLGAAMYGLVIGNIASLVSKLDAAKLLH RERMERVTAFLSYKKISPELQRRILEYFDYLWETRRGYEEREVLKELPHPLRLAVA MEIHGDVIEKVPLFKGAGEDFIRDIILHLEPVIYGPGEYIIRAGELGSDVYFINRGSVE VLSADEKTRYAILSEGQFFGEMALILRAPRTATVRARTFCDLYRLDKETFDRILSRY PEIAAQIQELA VRRKEELEGGTSRRGTGPGLKELACGSG (SEQ ID NO: 5). In some embodiments, the SthK protein consists of SEQ ID NO: 5. In some embodiments, SEQ ID NO: 1 encodes SEQ ID NO: 5.

In some embodiments, the channel is a homolog of SthK. In some embodiments, a homolog is encoded by a nucleotide sequence with at least 60%, at least 65%, at least 70%, at least 75%, at least 80%, at least 85%, at least 90%, at least 95%, at least 99% homology to the sequence presented in SEQ ID NO: 1. Each possibility represents a separate embodiment of the invention. In some embodiments, a homolog is encoded by a nucleotide sequence with at least 85% homology to SEQ ID NO: 1. In some embodiments, a sequence encoding SthK comprises a sequence with at least 85% homology to SEQ ID NO: 1. In some embodiments, a homolog comprises an amino acid sequence with at least 60%, at least 65%, at least 70%, at least 75%, at least 80%, at least 85%, at least 90%, at least 95%, at least 99% homology to the sequence presented in SEQ ID NO: 5. Each possibility represents a separate embodiment of the invention. In some embodiments, a homolog comprises at least 60%, at least 65%, at least 70%, at least 75%, at least 80%, at least 85%, at least 90%, at least 95%, at least 99% homology to the sequence presented in SEQ ID NO: 5. Each possibility represents a separate embodiment of the invention. In some embodiments, the homolog comprises at least 85% homology to SEQ ID NO: 5. In some embodiments, homology is identity.

In some embodiments, a homolog comprises SthK function. In some embodiments, SthK function comprises cyclic nucleotide gating. In some embodiments, the SthK function is potassium transport. The structure of SthK and its various domains have been well studied and are disclosed in Brams et al. (see above), Kesters et al., “Structure of the SthK carboxy-terminal region reveals a gating mechanism for cyclic nucleotide-modulated ion channels”, PLOS One, 2015 Jan. 27;10 (1): e0116369 and Nimigean and Rheinberger, “Structure of the SthK cyclic nucleotide-gated potassium channel in complex with CAMP”, rcsb.org/structure/6cju, all of which are incorporated herein by reference in their entirety. A skilled artisan being aware of the structure of SthK and the functional relationship between that structure and cAMP binding and potassium transport can determine which alterations to SEQ ID NO: 5 and thereby which alterations to SEQ ID NO: 1 would not adversely affect CAMP gating and potassium transport.

In some embodiments, the first sequence encodes a fragment of a cyclic nucleotide gated potassium channel. In some embodiments, the fragment is a functional fragment. In some embodiments, the function comprises a function of SthK. In some embodiments, the function comprises being cyclic nucleotide gated. In some embodiments, the function comprises cyclic nucleotide gating. In some embodiments, the function comprises being a potassium channel. In some embodiments, the function comprises potassium transport. In some embodiments, the function comprises potassium channel function.

In some embodiments, a fragment comprises at least 25, 30, 35, 40, 45, 50, 55, 60, 65, 70, 75, 80, 85, 90, 95, 100, 150, 200, 250, 300, 350, or 400 consecutive amino acids of the channel. Each possibility represents a separate embodiment of the invention. In some embodiments, a fragment comprises at least 50 consecutive amino acids of the channel. In some embodiments, a fragment comprises at least 100 consecutive amino acids of the channel. In some embodiments, a homolog is homologous to a fragment of the channel.

As used herein, the term “chemogenetic” refers to an engineered receptor that responds to a small molecule that is not endogenous to the environment of the receptor. In some embodiments, chemogenetically activatable comprises activatable by a small molecule. In some embodiments, the small molecule is a non-natural small molecule. In some embodiments, the small molecule is an artificial molecule. In some embodiments, the small molecule is a designer molecule. In some embodiments, the small molecule is synthetic molecule. In some embodiments, the small molecule is a ligand. In some embodiments, the ligand is a natural ligand. In some embodiments, the ligand is a naturally endogenous ligand not present at the target site. In some embodiments, not present at the target site comprises not present at concentrations necessary for activation. In some embodiments, the target site is a target cell. In some embodiments, the target site is a target tissue/organ. In some embodiments, the ligand is not a naturally endogenous ligand. In some embodiments, the ligand is not a natural ligand. In some embodiments, the ligand is not the natural ligand of the receptor. In some embodiments, the ligand is a synthetic ligand. In some embodiments, the small molecule is not endogenous to a subject or target cell. In some embodiments, the subject is a mammal. In some embodiments, the mammal is a human. In some embodiments, the target cell is a cardiac cell. In some embodiments, the cardiac cell is a cardiomyocyte. In some embodiments, the target cell is not a neuron. In some embodiments, the cell is a neuron. In some embodiments, the cell is a muscle cell. In some embodiments, the muscle is smooth muscle. In some embodiments, the muscle is striated muscle. In some embodiments, the cell is a pancreatic cell. In some embodiments, the target cell is an electrically active cell.

In some embodiments, the receptor generates cyclic nucleotides. In some embodiments, activation of the receptor generates cyclic nucleotides. In some embodiments, binding of the small molecule to the receptor generates cyclic nucleotides. In some embodiments, binding of the small molecule to an extracellular domain of the receptor generates cyclic nucleotides within a cell expressing the receptor. In some embodiments, activation of the receptor generates cAMP. In some embodiments, generates is produces.

In some embodiments, the receptor is a designer receptor exclusively activated by designer drugs (DREADD). In some embodiments, the receptor is a receptor activated solely by a synthetic ligand (RASSL). DREADDs are well known in the art and any such molecule may be used as part of the invention. In some embodiments, the DREADD is an excitatory DREADD. In some embodiments, the DREADD is G-protein coupled. In some embodiments, the G-protein is a Gas G-protein. In some embodiments, a Gas G-protein is a Gs G-protein. Examples of Gas coupled DREADD include but are not limited to GsD and rM3D. In some embodiments, the DREADD is derived from the M3 muscarinic receptor (M3). In some embodiments, the M3 is human M3 (hM3). In some embodiments, the M3 is murine M3 (mM3). In some embodiments, the M3 is rat M3 (rM3). The rat M3 receptor is disclosed in Entrez Gene ID number 1131. The mRNA encoding the hM3 can be found for example in RefSeq ID numbers NM_000740, NM_001347716, NM_001375978, NM_001375979, and NM_001375980. In some embodiments, the hM3 receptor is encoded by an mRNA comprising the nucleotide sequence atgaccttgcacagtaacagtacaacctcgcctttgtttcccaacatcagctcttcctgggtgcacagtccctcggaggcagggctg cccttggggacagtcactcagttgggcagctacaacatttcacaagaaactgggaatttctcctcaaacgacacctccagcgaccct ctcgggggtcacaccatctggcaagtggtcttcattgccttcttaaccggcttcctggcattggtgaccatcattggcaacatccttgt cattgtggccttcaaggtcaacaaacagctgaagacagtcaacaactacttcctcttaagcctggcctgtgcagacctgatcatcgg ggtcatttccatgaacctgttcactacctacatcattatgaaccgttgggcactggggaacttagcctgcgacctctggctttccattga ctatgtggccagcaatgcctctgtcatgaatctgctggtcatcagctttgacaggtacttttccatcactagaccactcacctaccgag ccaaaagaacaacaaaacgagctggtgtgatgattggtctggcttgggtcatctcctttgtcctatgggctcctgccatcttgttctgg caatactttgtagggaagagaactgtgcccccaggagaatgtttcattcagtttctgagtgagcccaccatcaccttcggcacggcg atcgctgccttttacatgcctgtcaccatcatgactattttatactggaggatctataaggaaactgagaagcgtaccaaagagctggc tggcctacaggcctctgggacagaagcggaggcagaaaactttgtccaccccacaggcagttctcgaagctgtagcagctatgaa ctgcaacagcaaggcgtgaaacgatcatccaggaggaagtacggtcgctgtcacttctggttcaccaccaagagctggaagccc agtgccgagcagatggaccaagaccacagcagcagcgacagttggaacaacaacgatgctgctgcctccctggaaaactctgct tcctccgatgaagaggacattggctcagagaccagggccatctattccattgtcctcaagcttccaggccatagctccatcctcaact ctaccaagctaccgtcctcagataacctgcaggtgtccaacgaggacctggggactgtggatgtggagagaaatgctcacaagct tcaggcccagaagagcatgggtgatggtgacaactgtcagaaggatttcaccaagcttcccatccagttagagtctgccgtggaca caggcaagacctctgacaccaactccteggcagacaagaccacggctactctacctctgtccttcaaggaggccacgctggctaa gaggtttgctctcaagaccagaagtcagatcaccaagcggaagaggatgtcgctcatcaaggagaagaaggccgcccagacgc tcagtgccatcttgctagccttcatcatcacgtggaccccctacaacatcatggtcctggtgaacaccttctgtgacagctgcataccc aaaacctattggaatctgggctactggctgtgctatatcaacagcaccgtgaaccctgtgtgctatgccctgtgcaacaaaacattca gaaccaccttcaagacgctcctcttgtgccagtgtgacaaaaggaagaggcgcaaacagcagtaccagcagagacagtcggtca tttttcacaagcgagtgccggaacaggccttg (SEQ ID NO: 9). In some embodiments, the hM3 receptor is encoded by an mRNA consisting of SEQ ID NO: 9. In some embodiments, the hM3 receptor comprises the amino acid sequence MTLHSNSTTSPLFPNISSSWVHSPSEAGLPLGTVTQLGSYNISQETGNFSSNDTSSDP LGGHTIWQVVFIAFLTGFLALVTIIGNILVIVAFKVNKQLKTVNNYFLLSLACADLII GVISMNLFTTYIIMNRWALGNLACDLWLSIDYVASNASVMNLL VISFDRYFSITRP LTYRAKRTTKRAGVMIGLAWVISFVLWAPAILFWQYFVGKRTVPPGECFIQFLSEP TITFGTAIAAFYMPVTIMTILYWRIYKETEKRTKELAGLQASGTEAEAENFVHPTGS SRSCSSYELQQQGVKRSSRRKYGRCHFWFTTKSWKPSAEQMDQDHSSSDSWNNN DAAASLENSASSDEEDIGSETRAIYSIVLKLPGHSSILNSTKLPSSDNLQVSNEDLGT VDVERNAHKLQAQKSMGDGDNCQKDFTKLPIQLESAVDTGKTSDTNSSADKTTA TLPLSFKEATLAKRFALKTRSQITKRKRMSLIKEKKAAQTLSAILLAFIITWTPYNIM VLVNTFCDSCIPKTYWNLGYWLCYINSTVNPVCYALCNKTFRTTFKTLLLCQCDK RKRRKQQYQQRQSVIFHKRVPEQAL (SEQ ID NO: 10). In some embodiments, the hM3 receptor consists of the amino acid sequence of SEQ ID NO: 10.

In some embodiments, the receptor is rM3D. In some embodiments, the receptor is the DREADD rM3D. In some embodiments, rM3D is encoded by a sequence comprising atgaccttgcacagtaacagtacaacctcgcctttgtttcccaacatcagctcttcctgggtgcacagtccctcggaggcagggctg cccttggggacagtcactcagttgggcagctacaacatttcacaagaaactgggaatttctcctcaaacgacacctccagcgaccct ctcgggggtcacaccatctggcaagtggtcttcattgccttcttaactggcttcctggcattggtgaccatcattggcaacatccttgtc attgtggccttcaaggtcaacaaacagctgaagacagtcaacaactacttcctcttaagcctggcctgtgcagacctgatcatcggg gtcatttccatgaacctgttcactacctacatcattatgaaccgttgggcactggggaacttagcctgcgacctctggctctccattga ctgtgtggccagcaatgcctctgtcatgaatctgctggtcatcagctttgacaggtacttttccatcacttctccattccgctaccagag cctgatgaccagggctcgagctggtgtgatgattggtctggcttgggtcatctcctttgtcctatgggctcctgccatcttgttctggca atactttgtagggaagagaactgtgcccccaggagaatgtttcattcagtttctgagtgagcccaccatcaccttcggcacggcgat cgctggcttttacatgcctgtcaccatcatgactattttatactggcgggtgtaccgggaggccaaggagcagatcaggaagatcga ccgctgcgagggccggttctatggcagccaggagcagccgcagccacccccgctcccccaacaccagcccatccteggcaac ggccgtgccagcaagaggaagacgtcccgtgtcatggccatgagggaacacaaagctctgcagacgctcagtgccatcttgctg gccttcatcatcacgtggaccccctacaacatcatggtcctggtgaacaccttctgtgacagctgcatacccaaaacctattggaatc tgggctactggctgtgctatatcaacagcaccgtgaaccctgtgtgctatgccctgtgcaacaaaacattcagaaccaccttcaaga cgctcctcttgtgccagtgtgacaaaaggaagaggcgcaaacagcagtaccagcagagacagtcggtcatttttcacaagcgagt gccggagcaggccttg (SEQ ID NO: 2). In some embodiments, rM3D is encoded by a sequence consisting of SEQ ID NO: 2. In some embodiments, rM3D protein comprises the amino acid sequence MTLHSNSTTSPLFPNISSSWVHSPSEAGLPLGTVTQLGSYNISQETGNFSSNDTSSDP LGGHTIWQVVFIAFLTGFLALVTIIGNILVIVAFKVNKQLKTVNNYFLLSLACADLII GVISMNLFTTYIIMNRWALGNLACDLWLSIDCVASNASVMNLLVISFDRYFSITSPF RYQSLMTRARAGVMIGLAWVISFVLWAPAILFWQYFVGKRTVPPGECFIQFLSEPT ITFGTAIAGFYMPVTIMTILYWRVYREAKEQIRKIDRCEGRFYGSQEQPQPPPLPQH QPILGNGRASKRKTSRVMAMREHKALQTLSAILLAFIITWTPYNIMVLVNTFCDSCI PKTYWNLGYWLCYINSTVNPVCYALCNKTFRTTFKTLLLCQCDKRKRRKQQYQQ RQSVIFHKRVPEQAL (SEQ ID NO: 6). In some embodiments, the rM3D protein consists of the amino acid sequence of SEQ ID NO: 6.

In some embodiments, the receptor is a homolog of rM3D. In some embodiments, a homolog is encoded by a nucleotide sequence with at least 60%, at least 65%, at least 70%, at least 75%, at least 80%, at least 85%, at least 90%, at least 95%, at least 99% homology to the sequence presented in SEQ ID NO: 2. Each possibility represents a separate embodiment of the invention. In some embodiments, a homolog is encoded by a nucleotide sequence with at least 85% homology to SEQ ID NO: 2. In some embodiments, a sequence encoding rM3D comprises a sequence with at least 85% homology to SEQ ID NO: 2. In some embodiments, a homolog comprises an amino acid sequence with at least 60%, at least 65%, at least 70%, at least 75%, at least 80%, at least 85%, at least 90%, at least 95%, at least 99% homology to the sequence presented in SEQ ID NO: 6. Each possibility represents a separate embodiment of the invention. In some embodiments, a homolog comprises at least 60%, at least 65%, at least 70%, at least 75%, at least 80%, at least 85%, at least 90%, at least 95%, at least 99% homology to the sequence presented in SEQ ID NO: 6. Each possibility represents a separate embodiment of the invention. In some embodiments, the homolog comprises at least 85% homology to SEQ ID NO: 6. In some embodiments, homology is identity.

In some embodiments, a homolog comprises rM3D function. In some embodiments, rM3D function comprises generating a cyclic nucleotide. In some embodiments, rM3D function comprises generating a cyclic nucleotide upon activation. In some embodiments, the rM3D function is chemogenetic activation. In some embodiments, the rM3D function is being chemogenetically activatable. The structure of rM3D and its various domains have been well studied and are disclosed in Guettier et al., “A chemical-genetic approach to study G protein regulation of B cell function in vivo”, PNAS, 2009 November 10;106 (45): 19197-202 and Farrell et al., “A Gas DREADD Mouse for Selective Modulation of cAMP Production in Striatopallidal Neurons”, Neuropsychopharmacology. 2013 April;38 (5): 854-62, both of which are incorporated herein by reference in their entirety. A skilled artisan being aware of the structure of rM3D and the functional relationship between that structure and cAMP production and synthetic ligand binding can determine which alterations to SEQ ID NO: 6 and thereby which alterations to SEQ ID NO: 2 would not adversely affect cAMP generation and synthetic ligand binding.

In some embodiments, the second sequence encodes a fragment of a chemogenetically activatable CAMP cyclic nucleotide generating receptor. In some embodiments, the fragment is a functional fragment. In some embodiments, the function comprises a function of rM3D. In some embodiments, the function comprises generating cyclic nucleotides. In some embodiments, the function comprises generating cyclic nucleotides upon activation. In some embodiments, the function comprises generating cyclic nucleotides upon synthetic ligand binding. In some embodiments, the function comprises being a chemogenetically activatable. In some embodiments, the function comprises binding a small molecule. In some embodiments, the function comprises synthetic ligand binding. In some embodiments, the function comprises activation by a synthetic ligand. In some embodiments, the function comprises activation by a synthetic ligand to produce cyclic nucleotides.

In some embodiments, a fragment comprises at least 25, 30, 35, 40, 45, 50, 55, 60, 65, 70, 75, 80, 85, 90, 95, 100, 150, 200, 250, 300, 350, or 400 consecutive amino acids of the receptor. Each possibility represents a separate embodiment of the invention. In some embodiments, a fragment comprises at least 50 consecutive amino acids of the receptor. In some embodiments, a fragment comprises at least 100 consecutive amino acids of the receptor. In some embodiments, a homolog is homologous to a fragment of the receptor.

In some embodiments, the nucleic acid molecule further comprises a third sequence. In some embodiments, the third sequence comprises an internal ribosome entry site (IRES). In some embodiments, the third sequence is between the first sequence and the second sequence and the first sequence comprises a stop codon. In some embodiments, the third sequence is between the first sequence and the second sequence and the first sequence is devoid of a stop codon. In some embodiments, the third sequence encodes a linker. In some embodiments, the linker is a peptide linker. In some embodiments, the sequence encoding a linker is between the first sequence and the second sequence. In some embodiments, the sequence encoding the linker is in the same reading frame as the first sequence and the second sequence. In some embodiments, the linker comprises at least 1, 2, 3, 4, 5, 6, 7, 8, 9, 10, 11, 12, 13, 14, 15, 16, 17, 18, 19, 20, 30, 40, 50, 60, 70, 80, 90, 100, 125, 150, 175, 200, 250, 300, 350, 400, 450 or 500 amino acids. Each possibility represents a separate embodiment of the invention. In some embodiments, the linker comprises at least 5 amino acids. In some embodiments, the linker comprises at least 300 amino acids. In some embodiments, the linker comprises at most 10, 15, 20, 25, 30, 35, 40, 45, 50, 60, 70, 80, 90, 100, 125, 150, 175, 200, 250, 300, 350, 400, 450 or 500 amino acids. Each possibility represents a separate embodiment of the invention. In some embodiments, the linker comprises at most 30 amino acids. In some embodiments, the linker comprises at most 400 amino acids. In some embodiments, the linker encodes an amino acid sequence which is not found in the receptor. In some embodiments, the linker encodes an amino acid sequence which is not found in the channel. In some embodiments, the amino acid sequence comprises at least 5 amino acids.

In some embodiments, the linker is a cleavable linker. In some embodiments, the linker is a self-cleaving linker. In some embodiments, the linker is a peptide. In some embodiments, the linker comprises a 2A self-cleaving peptide. In some embodiments, the linker consists of a 2A self-cleaving peptide. In some embodiments, the self-cleaving linker induces ribosome skipping. In some embodiments, the self-cleaving linker induces failure to make a peptide bond between an amino acid and the next amino acid to be produced by the open reading frame thus resulting in two separate proteins being produced. In some embodiments, the 2A peptide comprises the motif DX1EX2NPGP (SEQ ID NO: 13) wherein X1 is any amino acid and X2 is any amino acid. In some embodiments, SEQ ID NO: 13 is DVEENPGP (SEQ ID NO: 14). In some embodiments, the 2A peptide is selected from a T2A peptide, a P2A peptide, an E2A peptide and an F2A peptide. In some embodiments, the 2A peptide is a P2A peptide. In some embodiments, the linker comprises a P2A peptide. In some embodiments, the linker consists of a P2A peptide. In some embodiments, the P2A peptide is encoded by the sequence gccacgaacttctctctgttaaagcaagcaggagacgtggaagaaaaccccggtcct (SEQ ID NO: 3). In some embodiments, the linker is encoded by a sequence comprising SEQ ID NO: 3. In some embodiments, the linker is encoded by a sequence consisting of SEQ ID NO: 3. In some embodiments, the linker comprises the amino acid sequence ATNFSLLKQAGDVEENPGP (SEQ ID NO: 7). In some embodiments, the linker consists of the amino acid sequence of SEQ ID NO: 7. In some embodiments, the P2A linker comprises or consists of SEQ ID NO: 7.

In some embodiments, the nucleic acid molecule comprises SEQ ID NO: 4. In some embodiments, the nucleic acid molecule consists of SEQ ID NO: 4. In some embodiments, the open reading frame comprises SEQ ID NO: 4. In some embodiments, the open reading frame consists of SEQ ID NO: 4. In some embodiments, the nucleic acid molecule comprises a homolog of SEQ ID NO: 4. In some embodiments, the nucleic acid molecule consists of a homolog of SEQ ID NO: 4. In some embodiments, the open reading frame comprises a homolog of SEQ ID NO: 4. In some embodiments, the open reading frame consists of a homolog of SEQ ID NO: 4. In some embodiments, the homolog comprises at least 70, 80, 85, 90, 92, 95, 97 or 99% identity to SEQ ID NO: 4. Each possibility represents a separate embodiment of the invention. In some embodiments, the homolog comprises at least 85% identity to SEQ ID NO: 4. In some embodiments, the nucleic acid molecule encodes an auto-cleaving protein. In some embodiments, the auto-cleaving protein comprises MKSSAFSHPTYTLVWKVGILAVTLYYAIRIPLTLVFPSLFSPLLPLDILASLALIADIP LDFAFESRKTSGRKPTLLAPSRLPDLLAALPLDLLVFALHLPSPLSLLSLVRLLKLIS VQRSATRILSYRINPALLRLLSLVGFILLAAHGIACGWMSLQPPSESPAGTRYLSAF YWTITTLTTIGYGDITPSTPIQTVYTIVIELLGAAMYGLVIGNIASLVSKLDAAKLLH RERMERVTAFLSYKKISPELQRRILEYFDYLWETRRGYEEREVLKELPHPLRLAVA MEIHGDVIEKVPLFKGAGEDFIRDIILHLEPVIYGPGEYIIRAGELGSDVYFINRGSVE VLSADEKTRY AILSEGQFFGEMALILRAPRTATVRARTFCDLYRLDKETFDRILSRY PEIAAQIQELAVRRKEELEGGTSRRGTGPGLKELACGSGATNFSLLKQAGDVEENP GPMTLHSNSTTSPLFPNISSSWVHSPSEAGLPLGTVTQLGSYNISQETGNFSSNDTSS DPLGGHTIWQVVFIAFLTGFLALVTIIGNILVIVAFKVNKQLKTVNNYFLLSLACAD LIIGVISMNLFTTYIIMNRWALGNLACDLWLSIDCVASNASVMNLLVISFDRYFSITS PFRYQSLMTRARAGVMIGLAWVISFVLWAPAILFWQYFVGKRTVPPGECFIQFLSE PTITFGTAIAGFYMPVTIMTILYWRVYREAKEQIRKIDRCEGRFYGSQEQPQPPPLP QHQPILGNGRASKRKTSRVMAMREHKALQTLSAILLAFIITWTPYNIMVLVNTFCD SCIPKTYWNLGYWLCYINSTVNPVCYALCNKTFRTTFKTLLLCQCDKRKRRKQQY QQRQSVIFHKRVPEQAL (SEQ ID NO: 8). In some embodiments, the auto-cleaving protein consists of SEQ ID NO: 8. In some embodiments, the autocleaving protein comprises at least 70, 75, 80, 85, 90, 92, 95 or 97% homology or identity to SEQ ID NO: 8. Each possibility represents a separate embodiment of the invention. In some embodiments, the autocleaving protein comprises at least 85% identity to SEQ ID NO: 8. In some embodiments, the autocleaving protein retains potassium channel function. In some embodiments, the autocleaving protein retains being cyclic nucleotide gated. In some embodiments, the autocleaving protein retains chemogenetically activatable cyclic nucleotide generating function. In some embodiments, after autocleavage two proteins are produced, a first protein comprising the amino acid sequence MKSSAFSHPTYTLVWKVGILAVTLYYAIRIPLTLVFPSLFSPLLPLDILASLALIADIP LDFAFESRKTSGRKPTLLAPSRLPDLLAALPLDLLVFALHLPSPLSLLSLVRLLKLIS VQRSATRILSYRINPALLRLLSLVGFILLAAHGIACGWMSLOPPSESPAGTRYLSAF YWTITTLTTIGYGDITPSTPIQTVYTIVIELLGAAMYGLVIGNIASLVSKLDAAKLLH RERMERVTAFLSYKKISPELQRRILEYFDYLWETRRGYEEREVLKELPHPLRLAVA MEIHGDVIEKVPLFKGAGEDFIRDIILHLEPVIYGPGEYIIRAGELGSDVYFINRGSVE VLSADEKTRYAILSEGQFFGEMALILRAPRTATVRARTFCDLYRLDKETFDRILSRY PEIAAQIQELAVRRKEELEGGTSRRGTGPGLKELACGSGATNFSLLKQAGDVEENP G (SEQ ID NO: 15) and a second protein comprising the amino acid sequence PMTLHSNSTTSPLFPNISSSWVHSPSEAGLPLGTVTQLGSYNISQETGNFSSNDTSSD PLGGHTIWQVVFIAFLTGFLALVTIIGNILVIVAFKVNKQLKTVNNYFLLSLACADLI

IGVISMNLFTTYIIMNRWALGNLACDLWLSIDCVASNASVMNLLVISFDRYFSITSP FRYQSLMTRARAGVMIGLAWVISFVLWAPAILFWQYFVGKRTVPPGECFIQFLSEP TITFGTAIAGFYMPVTIMTILYWRVYREAKEQIRKIDRCEGRFYGSQEQPQPPPLPQ HQPILGNGRASKRKTSRVMAMREHKALQTLSAILLAFIITWTPYNIMVLVNTFCDS CIPKTYWNLGYWLCYINSTVNPVCYALCNKTFRTTFKTLLLCQCDKRKRRKQQY QQRQSVIFHKRVPEQAL (SEQ ID NO: 16). In some embodiments, the first protein consists of SEQ ID NO: 15. In some embodiments, the second protein consists of SEQ ID NO: 16.

In some embodiments, the nucleic acid molecule comprises the nucleotide sequence atgaaaagctccgccttctcccaccccacctacaccctggtctggaaagtcggcattctggctgtcactctgtattacgctattcgaat cccactgaccctggtgttcccctctctgtttagtcccctgctgcctctggatatcctggccagtctggctctgatcgcagacattcctct ggatttcgcctttgagtcacgaaagacaagcggcaggaaaccaactctgctggctcctagccgactgccagatctgctggccgct ctgccactggacctgctggtgttcgccctgcacctgccatcacccctgagcctgctgtccctggtgaggctgctgaagctgatctcc gtccagaggtctgctacaagaatcctgtcttacagaattaacccagcactgctgcggctgctgagtctggtgggattcatcctgctgg cagcccatgggattgcctgcggctggatgtcactgcagccacctagegagtccccagcaggaaccagatacctgagegccttcta ctggacaatcaccacactgactaccateggctacggagatattaccccatccacacccattcagaccgtgtacaccatcgtcattga gctgctgggagctgcaatgtatggactggtcatcgggaatattgcatctctggtcagtaagctggacgccgctaaactgctgcacc gagagaggatggaacgggtgacagctttcctgagttacaagaaaatctcacctgagctgcagaggagaattctggaatactttgatt atctgtgggagactcggcgcgggtatgaggaacgcgaggtgctgaaggaactgcctcacccactgcgactggctgtcgcaatgg aaatccatggcgacgtgattgagaaggtcccactgttcaaaggggccggcgaagactttatccgcgatatcattctgcatctggag cccgtgatctacggacctggggaatatatcattagggctggcgagctgggcagcgatgtctactttatcaacagaggcagcgtgga ggtcctgtccgcagacgaaaagacccggtatgccatcctgtctgagggccagttctttggagaaatggcactgattctgcgagcac cacgaacagctactgtgagagcacggactttctgtgacctgtacagactggataaagaaacctttgacagaatcctgtctcgctatc ctgagattgcagcccagattcaggaactggctgtgcggaggaaagaagaactggaaggggggacatcacggcggggaaccgg tcccgggcttaaggagctcgcatgcggaagcggaggacgtacggcggccaagagcaggatcaccagcgagggcgagtacat ccccctggaccagatcgacatcaacgtggcggtacccgtgagcaagggcgaggagctgttcaccggggtggtgcccatcctggt cgagctggacggcgacgtaaacggccacaagttcagcgtgtccggcgagggcgagggcgatgccacctacggcaagctgacc ctgaagttcatctgcaccaccggcaagctgcccgtgccctggcccaccctcgtgaccaccctgagctggggcgtgcagtgcttcg cccgctaccccgaccacatgaagcagcacgacttcttcaagtccgccatgcccgaaggctacgtccaggagegcaccatettctt caaggacgacggcaactacaagacccgcgccgaggtgaagttcgagggcgacaccctggtgaaccgcatcgagctgaaggg catcgacttcaaggaggacggcaacatcctggggcacaagctggagtacaacgccatccacggcaacgtctatatcaccgccga caagcagaagaacggcatcaaggccaacttcggcctcaactgcaacatcgaggacggcagegtgcagctcgccgaccactacc agcagaacacccccatcggcgacggccccgtgctgctgcccgacaaccactacctgagcacccagtccaagctgagcaaagac cccaacgagaagcgcgatcacatggtcctgctggagttcgtgaccgccgccgggatcactctcggcatggacgagctgtatacc ggtgcagccctgcaggagaagaagtcatgcagccagcgcatggccgaattccggcaatactgttggaacccggacactgggca gatgctgggccgcaccccagcccggtgggtgtggatcagcctgtactatgcagctttctacgtggtcatgactgggctctttgcctt gtgcatctatgtgctgatgcagaccattgatccctacacccccgactaccaggaccagttaaagtcaccgggggtaaccttgagac cggatgtgtatggggaaagagggctgcagatttcctacaacatctctgaaaacagctctagacaggcccagatcaccggacgtcc ggagggagctgtacaaatgaccttgcacagtaacagtacaacctcgcctttgtttcccaacatcagctcttcctgggtgcacagtcc ctcggaggcagggctgcccttggggacagtcactcagttgggcagctacaacatttcacaagaaactgggaatttctcctcaaacg acacctccagcgaccctctcgggggtcacaccatctggcaagtggtcttcattgccttcttaactggcttcctggcattggtgaccat cattggcaacatccttgtcattgtggccttcaaggtcaacaaacagctgaagacagtcaacaactacttcctcttaagcctggcctgt gcagacctgatcatcggggtcatttccatgaacctgttcactacctacatcattatgaaccgttgggcactggggaacttagcctgcg acctctggctctccattgactgtgtggccagcaatgcctctgtcatgaatctgctggtcatcagctttgacaggtacttttccatcacttc tccattccgctaccagagcctgatgaccagggctcgagctggtgtgatgattggtctggcttgggtcatctcctttgtcctatgggctc ctgccatcttgttctggcaatactttgtagggaagagaactgtgcccccaggagaatgtttcattcagtttctgagtgagcccaccatc accttcggcacggcgatcgctggcttttacatgcctgtcaccatcatgactattttatactggcgggtgtaccgggaggccaaggag cagatcaggaagatcgaccgctgcgagggccggttctatggcagccaggagcagccgcagccacccccgctcccccaacacc agcccatcctcggcaacggccgtgccagcaagaggaagacgtcccgtgtcatggccatgagggaacacaaagctctgcagacg ctcagtgccatcttgctggccttcatcatcacgtggaccccctacaacatcatggtcctggtgaacaccttctgtgacagctgcatac ccaaaacctattggaatctgggctactggctgtgctatatcaacagcaccgtgaaccctgtgtgctatgccctgtgcaacaaaacatt cagaaccaccttcaagacgctcctcttgtgccagtgtgacaaaaggaagaggcgcaaacagcagtaccagcagagacagtcggt catttttcacaagcgagtgccggagcaggccttg (SEQ ID NO: 27). In some embodiments, the nucleic acid molecule consists of SEQ ID NO: 27. In some embodiments, the open reading frame comprises SEQ ID NO: 27. In some embodiments, the open reading frame consists of SEQ ID NO: 27. In some embodiments, the nucleic acid molecule comprises a homolog of SEQ ID NO: 27. In some embodiments, the nucleic acid molecule consists of a homolog of SEQ ID NO: 27. In some embodiments, the open reading frame comprises a homolog of SEQ ID NO: 27. In some embodiments, the open reading frame consists of a homolog of SEQ ID NO: 27. In some embodiments, the homolog comprises at least 85% identity to SEQ ID NO: 27.

By another aspect, there is provided a vector comprising a nucleic acid molecule of the invention.

In some embodiments, the nucleic acid molecule is operatively linked to at least one transcriptional regulatory element. In some embodiments, the nucleic acid molecule further comprises at least one transcriptional regulatory element. In some embodiments, the transcriptional regulatory element is operatively linked to the open reading frame, the first open reading frame, the second open reading frame or a combination thereof. Each possibility represents a separate embodiment of the invention. In some embodiments, the transcriptional regulatory clement comprises a promoter. In some embodiments, the transcriptional regulatory clement is a promoter.

In some embodiments, the vector is an expression vector. The term “expression” as used herein refers to the biosynthesis of a gene product, including the transcription and/or translation of said gene product. Thus, expression of a nucleic acid molecule may refer to transcription of the nucleic acid fragment (e.g., transcription resulting in mRNA or other functional RNA) and/or translation of RNA into a precursor or mature protein (polypeptide). In some embodiments, expression is transcription of the gene product.

Expressing of a gene within a cell is well known to one skilled in the art. It can be carried out by, among many methods, transfection, viral infection, or direct alteration of the cell's genome. In some embodiments, the gene is in an expression vector such as plasmid or viral vector. One such example of an expression vector containing the nucleic acid molecule of the invention is the mammalian expression vector UCOE available from many retailers including Sigma Aldrich and Merck Millipore.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector, a lentiviral vector, or a poxviral vector. The promoters may be active in mammalian cells. The promoters may be a viral promoter. In some embodiments, the promoter is a constitutive promoter. In some embodiments, the vector is selected from: a mammalian expression vector, a lentiviral vector, an adeno-associated vector and any combination thereof.

In some embodiments, the promoter is active in cardiac cells. In some embodiments, the promoter is specifically active in cardiac cells. In some embodiments, the promoter is not active in non-cardiac cells. In some embodiments, specific expression or activity is expression or activity only in the target location (i.e., target cell). In some embodiments, the promoter is muscle specific. In some embodiments, the promoter is a muscle promoter. In some embodiments, the muscle is smooth muscle. In some embodiments, smooth muscles are cystic smooth muscles. In some embodiments, the muscle is striated muscle. In some embodiments, the muscle is cardiac muscle. In some embodiments, the promoter is cardiac specific. In some embodiments, the promoter is atrium specific. In some embodiments, the promoter is ventricle specific. In some embodiments, the cardiac cells are cardiomyocytes. In some embodiments, the promoter is a cardiac specific promoter. In some embodiments, the promoter is a cardiomyocyte specific promoter. In some embodiments, the at least one transcriptional regulatory clement comprises at least one cardiac cell enhancer. In some embodiments, the enhancer is a cardiac cell specific enhancer. In some embodiments, the promoter is a neuronal promoter. In some embodiments, the promoter is neuron specific. In some embodiments, the neuron is selected from: sensory neurons, motor neurons, interneurons, relay neurons, cholinergic neurons, adrenergic neurons, GABAergic neurons, glutamatergic neurons, dopaminergic neurons, serotonergic neurons, purinergic neurons and histaminergic neurons. In some embodiments, the promoter is a pancreatic promoter. In some embodiments, the promoter is pancreas specific. In some embodiments, the promoter is islet specific. In some embodiments, the promoter is beta cell specific. In some embodiments, the promoter is alpha cell specific. In some embodiments, the promoter is delta cell specific. In some embodiments, the promoter is acinar cell specific. In some embodiments, the promoter is pancreas ductal cell specific. In some embodiments, the promoter is a prostate promoter. In some embodiments, the promoter is prostate specific.

In some embodiments, the open reading frame is operably linked to a promoter. The term “operably linked” is intended to mean that the nucleotide sequence of interest is linked to the regulatory element or elements in a manner that allows for expression of the nucleotide sequence (e.g., in an in vitro transcription/translation system or in a host cell when the vector is introduced into the host cell).

In some embodiments, the vector is introduced into the cell by standard methods including electroporation (e.g., as described in From et al., Proc. Natl. Acad. Sci. USA 82, 5824 (1985)), heat shock, infection by viral vectors, high velocity ballistic penetration by small particles with the nucleic acid either within the matrix of small beads or particles, or on the surface (Klein et al., Nature 327. 70-73 (1987)), encapsulation within lipid particles, and/or the like.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (±), pGL3, pZeoSV2 (±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-IMTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

By another aspect, there is provided a kit comprising a cyclic nucleotide gated potassium channel or a fragment thereof and a chemogenetically activatable cyclic nucleotide generating receptor or a fragment thereof.

By another aspect, there is provided a fusion protein comprising a cyclic nucleotide gated potassium channel or a fragment thereof and a chemogenetically activatable cyclic nucleotide generating receptor or a fragment thereof.

In some embodiments, the fusion protein is a chimeric channel. In some embodiments, the fusion protein is a potassium selective chimeric channel. In some embodiments, the fusion protein is a cyclic nucleotide gated chimeric channel. In some embodiments, the fusion protein comprises a linker between the channel and the receptor. In some embodiments, the fusion protein is devoid of a linker. In some embodiments, the receptor is N-terminal to the channel. In some embodiments, the channel is N-terminal to the receptor. In some embodiments, the fusion protein further comprises a signal peptide. In some embodiments, the signal peptide is the signal peptide of the channel. In some embodiments, the signal peptide is the signal peptide of the receptor. In some embodiments, the N-terminal region of the fusion protein is the channel and comprises an N-terminal signal peptide and the C-terminal section is the receptor and does not comprise a signal peptide. In some embodiments, the N-terminal region of the fusion protein is the receptor and comprises an N-terminal signal peptide and the C-terminal section is the channel and does not comprise a signal peptide.

In some embodiments, the fragment is a functional fragment. In some embodiments, the fragment is functional within the fusion protein. In some embodiments, the fusion protein is encoded by a nucleic acid molecule of the invention. In some embodiments, the fusion protein is encoded by a vector of the invention.

In some embodiments, the cyclic nucleotide gated potassium channel is SthK. In some embodiments, the chemogenetically activatable cyclic nucleotide generating receptor is rM3D. In some embodiments, the fusion protein is a SthK-rM3D fusion. In some embodiments, the fusion protein is a fusion of SthK and rM3D. In some embodiments, the fusion protein comprises full length SthK. In some embodiments, the fusion protein comprises SEQ ID NO: 5. In some embodiments, the fusion protein comprises a functional fragment of SEQ ID NO: 5. In some embodiments, the fusion protein comprises a homolog of SEQ ID NO: 5. In some embodiments, the fusion protein comprises full length rM3D. In some embodiments, the fusion protein comprises SEQ ID NO: 6. In some embodiments, the fusion protein comprises a functional fragment of SEQ ID NO: 6. In some embodiments, the fusion protein comprises a homolog of SEQ ID NO: 6. In some embodiments, the fusion protein comprises SEQ ID NO: 5 and SEQ ID NO: 6 or homologs, fragments or derivatives thereof. In some embodiments, the fusion protein comprises SEQ ID NO: 5 and SEQ ID NO: 6.

In some embodiments, the fusion protein comprises a linker. In some embodiments, the linker is between the cyclic nucleotide gated potassium channel and the chemogenetically activable cyclic nucleotide generating receptor. In some embodiments, the fusion protein comprises a spacer. In some embodiments, the spacer is between the cyclic nucleotide gated potassium channel and the chemogenetically activable cyclic nucleotide generating receptor. In some embodiments, SthK and rM3D are linked by a linker. In some embodiments, the SthK and rM3D are separated by a spacer. In some embodiments, the C-terminus of SthK is connected to the linker or spacer. In some embodiments, the N-terminus of SthK is connected to the linker or spacer. In some embodiments, the C-terminus of rM3D is connected to the linker or spacer. In some embodiments, the N-terminus of rM3D is connected to the linker or spacer.

The selection of a linker/spacer is not trivial as both the receptor and channel are transmembrane proteins and must be properly inserted into the plasma membrane in order to function. That is, the extracellular domains of both the receptor and channel must be in the extracellular space when the fusion protein is expressed in a plasma membrane. In some embodiments, the spacer and/or linker is of a sufficient length such that the channel and receptor are both expressed in the correct orientation in the plasma membrane.

In some embodiments, the linker and/or spacer encodes an amino acid sequence which is not found in the receptor. In some embodiments, the linker and/or spacer encodes an amino acid sequence which is not found in the channel. In some embodiments, the amino acid sequence comprises at least 5 amino acids. In some embodiments, the spacer comprises GRTAAKSRITSEGEYIPLDQIDINVAVP (SEQ ID NO: 18). In some embodiments, the spacer consists of SEQ ID NO: 18. In some embodiments, the spacer is encoded by the nucleotide sequence Ggacgtacggcggccaagagcaggatcaccagcgagggcgagtacatccccctggaccagatcgacatcaacgtggcggta ccc (SEQ ID NO: 17).

In some embodiments, the spacer is a fluorescent spacer. In some embodiments, the spacer comprises a fluorescent protein. Examples of fluorescent proteins include, but are not limited to GFP, YFP, RFP, Cerulean, Cy5, and Cy7. In some embodiments, the fluorescent protein is mCerulean3. In some embodiments, the spacer comprises VSKGEELFTGVVPILVELDGDVNGHKFSVSGEGEGDATYGKLTLKFICTTGKLPVP WPTLVTTLSWGVQCFARYPDHMKQHDFFKSAMPEGYVQERTIFFKDDGNYKTRA EVKFEGDTLVNRIELKGIDFKEDGNILGHKLEYNAIHGNVYITADKQKNGIKANFG LNCNIEDGSVQLADHYQQNTPIGDGPVLLPDNHYLSTQSKLSKDPNEKRDHMVLL EFVTAAGITLGMDELY (SEQ ID NO: 20). In some embodiments, mCerulean3 comprises SEQ ID NO: 20. In some embodiments, the spacer consists of SEQ ID NO: 20. In some embodiments, mCerulean3 consists of SEQ ID NO: 20. In some embodiments, the spacer is encoded by a nucleotide sequence comprising gtgagcaagggcgaggagctgttcaccggggtggtgcccatcctggtcgagctggacggcgacgtaaacggccacaagttcag cgtgtccggcgagggcgagggcgatgccacctacggcaagctgaccctgaagttcatctgcaccaccggcaagctgcccgtgc cctggcccaccctcgtgaccaccctgagctggggcgtgcagtgcttegcccgctaccccgaccacatgaagcagcacgacttctt caagtccgccatgcccgaaggctacgtccaggagcgcaccatcttcttcaaggacgacggcaactacaagacccgcgccgagg tgaagttcgagggcgacaccctggtgaaccgcatcgagctgaagggcatcgacttcaaggaggacggcaacatcctggggcac aagctggagtacaacgccatccacggcaacgtctatatcaccgccgacaagcagaagaacggcatcaaggccaacttcggcctc aactgcaacatcgaggacggcagcgtgcagctcgccgaccactaccagcagaacacccccatcggcgacggccccgtgctgct gcccgacaaccactacctgagcacccagtccaagctgagcaaagaccccaacgagaagcgcgatcacatggtcctgctggagtt cgtgaccgccgccgggatcactctcggcatggacgagetgtat (SEQ ID NO: 19). In some embodiments, the spacer is encoded by a nucleotide sequence consisting of SEQ ID NO: 19. In some embodiments, the mCerulean3 is encoded by SEQ ID NO: 19. In some embodiments, SEQ ID NO: 19 encodes SEQ ID NO: 20. In some embodiments, the linker is derived from a natural protein. In some embodiments, the linker comprises a fragment of a natural protein. In some embodiments, the linker is derived from an ion transporting ATPase. In some embodiments, the linker is derived from an ion transporting ATPase subunit. In some embodiments, derived from is a fragment of. In some embodiments, the linker is a functional a ion-transporting ATPase. In some embodiments, the linker is derived from sodium/potassium-transporting ATPase. In some embodiments, the linker is derived from a potassium-transporting ATPase. In some embodiments, the linker is derived from a potassium-transporting ATPase subunit. In some embodiments, the linker is derived from a potassium-transporting ATPase subunit beta. In some embodiments, the potassium-transporting ATPase subunit beta is from rat. In some embodiments, the rat potassium-transporting ATPase subunit beta is provided in RefSeq number NP_036642. In some embodiments, the rat potassium-transporting ATPase subunit beta is encoded by an mRNA provided in RefSeq number NP_012510. In some embodiments, the derivative is a fragment from the rat potassium-transporting ATPase subunit beta. In some embodiments, the fragment comprises at least the first 100 amino acids. In some embodiments, the linker comprises AALQEKKSCSQRMAEFRQYCWNPDTGQMLGRTPARWVWISLYYAAFYVVMTGL FALCIYVLMQTIDPYTPDYQDQLKSPGVTLRPDVYGERGLQISYNISENS (SEQ ID NO: 22). In some embodiments, the rat potassium-transporting ATPase subunit beta comprises SEQ ID NO: 22. In some embodiments, the linker consists of SEQ ID NO: 22. In some embodiments, the fragment of rat potassium-transporting ATPase subunit beta consists of SEQ ID NO: 22. In some embodiments, the rat potassium-transporting ATPase subunit beta is N-terminal to the receptor. In some embodiments, the rat potassium-transporting ATPase subunit beta is C-terminal to the channel. In some embodiments, the rat potassium-transporting ATPase subunit beta fragment is encoded by a nucleotide sequence comprising Gcagccctgcaggagaagaagtcatgcagccagcgcatggccgaattccggcaatactgttggaacccggacactgggcagat gctgggccgcaccccagcccggtgggtgtggatcagcctgtactatgcagctttctacgtggtcatgactgggctctttgccttgtg catctatgtgctgatgcagaccattgatccctacacccccgactaccaggaccagttaaagtcaccgggggtaaccttgagaccgg atgtgtatggggaaagagggctgcagatttcctacaacatctctgaaaacagc (SEQ ID NO: 21). In some embodiments, the rat potassium-transporting ATPase subunit beta fragment is encoded by a nucleotide sequence consisting of SEQ ID NO: 21. In some embodiments, the linker is derived from SEQ ID NO: 22. In some embodiments, a sequence derived from SEQ ID NO: 22 is a homolog to SEQ ID NO: 22.

In some embodiments, the linker comprises SRQAQITGRPEGAVQ (SEQ ID NO: 24). In some embodiments, the linker consists of SEQ ID NO: 24. In some embodiments, the linker is encoded by a nucleotide sequence comprising Tctagacaggcccagatcaccggacgtccggagggagctgtacaa (SEQ ID NO: 23). In some embodiments, the linker is encoded by a nucleotide sequence consisting of SEQ ID NO: 23. In some embodiments, the linker and/or spacer comprises the amino acid sequence GRTAAKSRITSEGEYIPLDQIDINVAVPVSKGEELFTGVVPILVELDGDVNGHKFSV SGEGEGDATYGKLTLKFICTTGKLPVPWPTLVTTLSWGVQCFARYPDHMKQHDFF KSAMPEGYVQERTIFFKDDGNYKTRAEVKFEGDTLVNRIELKGIDFKEDGNILGHK LEYNAIHGNVYITADKQKNGIKANFGLNCNIEDGSVQLADHYQQNTPIGDGPVLLP DNHYLSTQSKLSKDPNEKRDHMVLLEFVTAAGITLGMDELYTGAALQEKKSCSQ RMAEFRQYCWNPDTGQMLGRTPARWVWISLYYAAFYVVMTGLFALCIYVLMQT IDPYTPDYQDQLKSPGVTLRPDVYGERGLQISYNISENSSRQAQITGRPEGAVQ (SEQ ID NO: 26). In some embodiments, the linker and/or spacer consists of SEQ ID NO: 26. In some embodiments, the linker and/or spacer is derived from SEQ ID NO: 26. In some embodiments, SEQ ID NO: 26 is encoded by a nucleotide sequence comprising Ggacgtacggcggccaagagcaggatcaccagcgagggcgagtacatccccctggaccagatcgacatcaacgtggcggta cccgtgagcaagggcgaggagctgttcaccggggtggtgcccatcctggtcgagctggacggcgacgtaaacggccacaagtt cagcgtgtccggcgagggcgagggcgatgccacctacggcaagctgaccctgaagttcatctgcaccaccggcaagctgcccg tgccctggcccaccctcgtgaccaccctgagctggggcgtgcagtgcttcgcccgctaccccgaccacatgaagcagcacgact tcttcaagtccgccatgcccgaaggctacgtccaggagcgcaccatcttcttcaaggacgacggcaactacaagacccgcgccg aggtgaagttcgagggcgacaccctggtgaaccgcatcgagctgaagggcatcgacttcaaggaggacggcaacatcctgggg cacaagctggagtacaacgccatccacggcaacgtctatatcaccgccgacaagcagaagaacggcatcaaggccaacttcgg cctcaactgcaacatcgaggacggcagcgtgcagctcgccgaccactaccagcagaacacccccatcggcgacggccccgtg ctgctgcccgacaaccactacctgagcacccagtccaagctgagcaaagaccccaacgagaagcgcgatcacatggtcctgctg gagttcgtgaccgccgccgggatcactctcggcatggacgagctgtataccggtgcagccctgcaggagaagaagtcatgcagc cagcgcatggccgaattccggcaatactgttggaacccggacactgggcagatgctgggccgcaccccagcccggtgggtgtg gatcagcctgtactatgcagctttctacgtggtcatgactgggctctttgccttgtgcatctatgtgctgatgcagaccattgatccctac acccccgactaccaggaccagttaaagtcaccgggggtaaccttgagaccggatgtgtatggggaaagagggctgcagatttcct acaacatctctgaaaacagctctagacaggcccagatcaccggacgtccggagggagctgtacaa (SEQ ID NO: 25). In some embodiments, SEQ ID NO: 26 is encoded by a nucleotide sequence consisting of SEQ ID NO: 25.

In some embodiments, the fusion protein comprises the amino acid sequence MKSSAFSHPTYTLVWKVGILAVTLYYAIRIPLTLVFPSLFSPLLPLDILASLALIADIP LDFAFESRKTSGRKPTLLAPSRLPDLLAALPLDLLVFALHLPSPLSLLSLVRLLKLIS VQRSATRILSYRINPALLRLLSLVGFILLAAHGIACGWMSLOPPSESPAGTRYLSAF YWTITTLTTIGYGDITPSTPIQTVYTIVIELLGAAMYGLVIGNIASLVSKLDAAKLLH RERMERVTAFLSYKKISPELQRRILEYFDYLWETRRGYEEREVLKELPHPLRLAVA MEIHGDVIEKVPLFKGAGEDFIRDIILHLEPVIYGPGEYIIRAGELGSDVYFINRGSVE VLSADEKTRYAILSEGQFFGEMALILRAPRTATVRARTFCDLYRLDKETFDRILSRY PEIAAQIQELAVRRKEELEGGTSRRGTGPGLKELACGSGGRTAAKSRITSEGEYIPL DQIDINVAVPVSKGEELFTGVVPILVELDGDVNGHKFSVSGEGEGDATYGKLTLKF ICTTGKLPVPWPTLVTTLSWGVQCFARYPDHMKQHDFFKSAMPEGYVQERTIFFK DDGNYKTRAEVKFEGDTLVNRIELKGIDFKEDGNILGHKLEYNAIHGNVYITADK QKNGIKANFGLNCNIEDGSVQLADHYQQNTPIGDGPVLLPDNHYLSTQSKLSKDP NEKRDHMVLLEFVTAAGITLGMDELYTGAALQEKKSCSQRMAEFRQYCWNPDT GQMLGRTPARWVWISLYYAAFYVVMTGLFALCIYVLMQTIDPYTPDYQDQLKSP GVTLRPDVYGERGLQISYNISENSSRQAQITGRPEGA VQMTLHSNSTTSPLFPNISSS WVHSPSEAGLPLGTVTQLGSYNISQETGNFSSNDTSSDPLGGHTIWQVVFIAFLTGF LALVTIIGNILVIVAFKVNKQLKTVNNYFLLSLACADLIIGVISMNLFTTYIIMNRWA LGNLACDLWLSIDCVASNASVMNLLVISFDRYFSITSPFRYQSLMTRARAGVMIGL AWVISFVLWAPAILFWQYFVGKRTVPPGECFIQFLSEPTITFGTAIAGFYMPVTIMTI LYWRVYREAKEQIRKIDRCEGRFYGSQEQPQPPPLPQHQPILGNGRASKRKTSRVM AMREHKALQTLSAILLAFIITWTPYNIMVLVNTFCDSCIPKTYWNLGYWLCYINST VNPVCYALCNKTFRTTFKTLLLCQCDKRKRRKQQYQQRQSVIFHKRVPEQAL (SEQ ID NO: 28). In some embodiments, the fusion protein consists of SEQ ID NO: 28. In some embodiments, the nucleic acid molecule comprises a sequence encoding SEQ ID NO: 28. In some embodiments, the fusion protein comprises a homolog of SEQ ID NO: 28. In some embodiments, the fusion protein consists of a homolog of SEQ ID NO: 28. In some embodiments, the homolog comprises at least 85% identity to SEQ ID NO: 28. In some embodiments, the nucleic acid molecule comprises SEQ ID NO: 27. In some embodiments, the nucleic acid molecule consists of SEQ ID NO: 27. In some embodiments, the open reading frame comprises SEQ ID NO: 27. In some embodiments, the open reading frame consists of SEQ ID NO: 27. In some embodiments, SEQ ID NO: 27 encodes SEQ ID NO: 28.

By another aspect, there is provided a cell comprising a nucleic acid molecule of the invention. By another aspect, there is provided a cell comprising a vector of the invention. By another aspect, there is provided a cell comprising a fusion protein of the invention.

In some embodiments, the cell is a cardiac cell. In some embodiments, the cell is a cardiomyocyte. In some embodiments, the cell is a cell for adoptive cell transplant. In some embodiments, the cell is an induced pluripotent stem cell (iPSC). In some embodiments, the cell is an induced cardiac cell. In some embodiments, the cell is an iPSC differentiated to a cardiac cell.

In some embodiments, the cell is a mammalian cell. In some embodiments, the cell is a human cell. In some embodiments, the cell is not a neuron. In some embodiments, the cell is a neuron. In some embodiments, the cell is a muscle cell. In some embodiments, the cell is an electrically activatable cell. In some embodiments, the cell is a pancreatic cell. In some embodiments, the cell is a prostate cell. In some embodiments, the cell is a cell of a subject. In some embodiments, the cell is allogeneic to the subject. In some embodiments, the cell is autologous to the subject. In some embodiments, the cell is syngeneic to the subject. In some embodiments, the cell is suitable for adoptive cell transfer.

In some embodiments, the cell is an electrically active cell. As used herein, the term “electrically active cell” refers to a cell whose function is determined by the generation or reception of an electrical signal. In some embodiments, the cell is a non-neuronal electrically active cell. In some embodiments, the electrically active cell is an electrically responsive cell. In some embodiments, the electrically active cell is an electrically signaling cell. In some embodiments, the cell is a pacemaker cell. In some embodiments, the cardiac cell is a pacemaker cell.

By another aspect, there is provided a composition comprising a nucleic acid molecule of the invention. By another aspect, there is provided a composition comprising a vector of the invention. By another aspect, there is provided a composition comprising a fusion protein of the invention. By another aspect, there is provided a composition comprising a cell of the invention.

In some embodiments, the composition is a therapeutic composition. In some embodiments, the composition is a pharmaceutical composition. In some embodiments, the composition further comprises a pharmaceutically acceptable carrier excipient or adjuvant.

As used herein, the term “carrier,” “adjuvant” or “excipient” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

In some embodiments, the composition comprises a therapeutically effective amount of the nucleic acid molecule of the invention. In some embodiments, the composition comprises a therapeutically effective amount of the vector of the invention. In some embodiments, the composition comprises a therapeutically effective amount of the fusion protein of the invention. In some embodiments, the composition comprises a therapeutically effective amount of the cells of the invention. In some embodiments, an effective amount is an amount sufficient to induce hyperpolarization of a cell comprising the nucleic acid molecule, vector or fusion protein of the invention when it is contacted with a ligand of the receptor. In some embodiments, an effective amount is an amount sufficient to induce hyperpolarization of a cell adjacent to a cell of the invention when the cell of the invention is contacted with a ligand of the receptor. In some embodiments, an effective amount is an amount sufficient to treat a disease or condition in a subject in need thereof, wherein the subject is administered a ligand of the receptor.

In some embodiments, the composition is for use in hyperpolarizing a cell. In some embodiments, the composition is for use in treating a disease or condition. In some embodiments, the nucleic acid molecule is for use in hyperpolarizing a cell. In some embodiments, the nucleic acid molecule is for use in treating a disease or condition. In some embodiments, the vector is for use in hyperpolarizing a cell. In some embodiments, the vector is for use in treating a disease or condition. In some embodiments, the fusion protein is for use in hyperpolarizing a cell. In some embodiments, the fusion protein is for use in treating a disease or condition. In some embodiments, the treating is treating a subject. In some embodiments, the subject is in need of the treating. In some embodiments, the use is in combination with a ligand of the receptor. In some embodiments, the disease or condition is a cardiac disease or condition.

In some embodiments, the composition is formulated for administration to a subject. In some embodiments, the composition is formulated for systemic administration. In some embodiments, the composition is formulated for administration to a heart. In some embodiments, the heart is a heart of a subject. In some embodiments, the composition is formulated for administration to cardiac cells in culture.

Therapeutic delivery of DNA and RNA constructs is well known in the art and any such method or composition which allows for such delivery is included. Nanoparticles and in particular lipid nanoparticles (LNPs) have been successfully used to deliver nucleic acid therapeutics and may be used in the methods of the invention. In some embodiments, the composition comprises a nanoparticle encapsulating a vector or nucleic acid molecule of the invention. In some embodiments, the nanoparticle is an LNP. In some embodiments, the nanoparticle comprises a cardiac targeting moiety.

In some embodiments, the administering results in at least 5, 10, 15, 20, 25, 30, 35, 40, 45, or 50% of target cells receiving the vector or nucleic acid molecule of the invention. Each possibility represents a separate embodiment of the invention. In some embodiments, the administering results in at least 5% of target cells receiving the vector or nucleic acid molecule of the invention. In some embodiments, the administering results in at least 10% of target cells receiving the vector or nucleic acid molecule of the invention. In some embodiments, the administering results in at least 25% of target cells receiving the vector or nucleic acid molecule of the invention. In some embodiments, receiving is expressing. In some embodiments, expressing is expression of RNA. In some embodiments, expression is expression of protein. In some embodiments, target cells are cardiac cells. In some embodiments, target cells are specific cells of the heart. In some embodiments, target cells are diseased cells. In some embodiments, target cells are arrhythmic cells.

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of a composition of the present subject matter to a patient in need thereof. In some embodiments, the administration is intravenous administration. In some embodiments, the administration is cardiac administration. In some embodiments, the administration is selected from intramyocardial, intrapericardial and intracoronary administration. In some embodiments, the administration is intramyocardial administration. Examples of administration methods which are site specific to the heart include, but are not limited to atrial painting and cardiac catheter mediated delivery. In some embodiments, the administration comprises atrial painting. In some embodiments, painting is painting cells of the invention. In some embodiments, painting is painting the composition of the invention. In some embodiments, cardiac administration comprises cardiac catheter mediated administration. Other suitable routes of administration can include parenteral, subcutaneous, oral, intramuscular, or intraperitoneal.

The dosage administered will be dependent upon the age, health, and weight of the recipient, kind of concurrent treatment, if any, frequency of treatment, and the nature of the effect desired.

By another aspect, there is provided a method of hyperpolarizing a cell, the method comprising expressing in the cell a nucleic acid of the invention thereby hyperpolarizing a cell. By another aspect, there is provided a method of hyperpolarizing a cell, the method comprising expressing in the cell a vector of the invention thereby hyperpolarizing a cell. By another aspect, there is provided a method of hyperpolarizing a cell, the method comprising expressing in the cell a fusion protein of the invention thereby hyperpolarizing a cell.

By another aspect, there is provided a method of treating or preventing a disease or condition, the method comprising administering to a subject a nucleic acid of the invention thereby treating or preventing disease or condition. By another aspect, there is provided a method of treating or preventing a disease or condition, the method comprising administering to a subject a vector of the invention thereby treating or preventing disease or condition. By another aspect, there is provided a method of treating or preventing a disease or condition, the method comprising administering to a subject a fusion protein of the invention thereby treating or preventing disease or condition.

In some embodiments, the method is a method of treatment. In some embodiments, the method is a method of prevention. As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition or method herein needs only to reduce the severity of a disease, disorder, or condition, reduce the severity of symptoms associated therewith, or provide improvement to a patient or subject's quality of life.

In some embodiments, the treating is treating a subject. In some embodiments, the preventing is preventing in a subject. In some embodiments, the subject suffers from a cardiac disease or condition. In some embodiments, the subject is in need of treatment. In some embodiments, the subject is at risk of developing the disease or condition. In some embodiments, the subject is a human. In some embodiments, the subject does not suffer from a neurological disease, disorder or condition.

In some embodiments, method further comprises contacting the cell with a ligand. In some embodiments, method further comprises administering a ligand to the subject. In some embodiments, administering a ligand is administering a composition comprising the ligand. In some embodiments, the composition is a pharmaceutical composition. In some embodiments, the composition is formulated for systemic administration. In some embodiments, the ligand is a ligand of the receptor. In some embodiments, the ligand is a synthetic ligand. In some embodiments, the ligand is an artificial ligand. In some embodiments, the ligand is a ligand of the chemogenetically activatable cyclic nucleotide generating receptor. In some embodiments, the ligand is a naturally endogenous ligand not present at the target site. In some embodiments, not present at the target site comprises not present at concentrations necessary for activation. In some embodiments, the target site is a target cell. In some embodiments, the target site is a target tissue/organ. In some embodiments, the ligand is not a naturally endogenous ligand. In some embodiments, the ligand is not a naturally occurring ligand. In some embodiments, the ligand is not the natural ligand of the receptor. In some embodiments, the ligand does not bind to any naturally occurring receptor. In some embodiments, the ligand does not bind to any receptor endogenous to the cell. In some embodiments, the ligand does not bind to any receptor endogenous to the subject. In some embodiments, the ligand is clozapine-N-oxide (CNO). In some embodiments, the ligand is DREADD agonist 21. In some embodiments, DREADD agonist 21 is compound 21 (C21). In some embodiments, the ligand is Varenicline. Synthetic ligands and their matched receptors are well known in the art and any such ligand may be used. Examples of such ligands include, but are not limited to CNO, SalB, DCZ, Varenicline and C21.

In some embodiments, the disease or condition is characterized by electrical disfunction. In some embodiments, the disease or condition is characterized by electrical disfunction in a disease cell or tissue. In some embodiments, the disease or condition is characterized by abnormal electrical signaling. In some embodiments, the disease or condition is characterized by pathological electrical signaling. In some embodiments, characterized by is caused by. In some embodiments, the disease or condition is a disease or condition of an electrically active cell. In some embodiments, the disease or condition is a cardiac disease or condition. In some embodiments, the cardiac disease is an arrythmia. In some embodiments, the disease or condition comprises arrythmia. In some embodiments, the disease or condition comprises tachycardia. In some embodiments, the disease or condition comprises abnormal heart rhythm or sinus. In some embodiments, the cardiac disease or condition is selected from: arrhythmia, tachy-arrhythmia, brady-arrhythmia, bradycardia and tachycardia. It will be understood that tachyarrhythmias can be due to reentrant mechanisms or non-reentrant mechanisms. Similarly, focal arrhythmias can be due to abnormal automaticity or triggered activity. In some embodiments, tachyarrhythmias comprise atrial fibrillation (AF), atrial flutter, focal or reentrant atrial tachycardias, supraventricular arrhythmias, ventricular tachycardias, and ventricular fibrillation. Targeted bradyarrhythmias can include those resulting from abnormalities in initiating the electrical activity of the heart (sinus node dysfunction) and those that result from abnormalities in electrical conduction (various degrees of atrioventricular (AV) block).

In some embodiments, the disease or condition is a neurological disease or condition. In some embodiments, the disease or condition is disease or condition of neurons. In some embodiments, the neurological disease is characterized by hyperactivity of a neuron. In some embodiments, the neurological disease or condition is caused by hyperactivity of a neuron. In some embodiments, the neurological disease or condition is a neuronal hyperactivity disease or condition. In some embodiments, the neurological disease or condition is characterized by neuronal hyperactivity. In some embodiments, hyperactivity is hyperactivity of an excitatory neuron. In some embodiments, hyperactivity is hyperactivity of an inhibitory neuron. In some embodiments, the neurological disease or condition is selected from: epilepsy, Parkinson's and parkinsonian syndromes, essential tremor, restless leg syndrome, tinnitus, pain, and phantom sensations, Alzheimer's disease and neuropathy. In some embodiments, the neurological disease or condition is a neurodegenerative disease or condition.

In some embodiments, the disease or condition is a smooth muscle disease or condition. In some embodiments, the disease or condition is a disease or condition of the smooth muscle. In some embodiments, a smooth muscle disease or condition is a disease of the digestive tract. In some embodiments, a smooth muscle disease or condition is a prostate disease or condition. In some embodiments, a smooth muscle disease or condition is a circulatory disease or condition. In some embodiments, a smooth muscle disease or condition is a bladder disease or condition. In some embodiments, a smooth muscle disease or condition is selected from: benign prostatic hyperplasia (BPH), hypertension, erectile dysfunction, coronary artery disease, pathologies of the stomach and intestines leading to lack of motility or hyper motility, achalasia, gastroesophageal reflux disease (GERD), urinary incontinence and urinary retention.

In some embodiments, the disease or condition is a striated muscle disease or condition. In some embodiments, striated muscle is skeletal muscle. In some embodiments, a striated muscle disease or condition is a disease or condition requirement muscle relaxation.

In some embodiments, treating comprises ameliorating. In some embodiments, treating further comprises preventing. In some embodiments, treatment comprises returning a heart to normal sinus rhythm. In some embodiments, the treatment comprises returning heart cells adjacent to cells of the invention or cells expressing a nucleic acid molecule, vector or fusion protein of the invention to normal sinus rhythm. In some embodiments, treating comprises hyperpolarization of cardiac cells. In some embodiments, treatment comprises temporary cessation of electrical signaling in cardiac cells. In some embodiments, treatment comprises cessation of an arrythmia. In some embodiments, treatment occurs upon administration of the ligand.

By another aspect, there is provided a kit comprising:
a. a nucleic acid molecule of the invention, vector of the invention, fusion protein of the invention, cell of the invention, or composition of the invention; and
b. a ligand of the chemogenetically activatable cyclic nucleotide generating receptor.

In some embodiments, the kit is for use in a method of the invention. In some embodiments, the kit is for use in hyperpolarizing a cell. In some embodiments, the cell is a target cell. In some embodiments, the kit is for use in treating a cardiac disease or condition. In some embodiments, the kit further comprises instructions. In some embodiments, the instructions are instructions for performing a method of the invention. In some embodiments, the instructions indicate the ligand is for use with the receptor. In some embodiments, the instructions indicate the ligand is for use with the nucleic acid molecule. In some embodiments, the instructions indicate the ligand is for use in combination. In some embodiments, the kit further comprises labels. In some embodiments, the labels indicate the ligand is for use in combination. In some embodiments, in combination is in combination with the receptor. In some embodiments, in combination is in combination with the nucleic acid molecule. In some embodiments, in combination is in combination with the vector. In some embodiments, in combination is in combination with the fusion protein. In some embodiments, in combination is in combination with the cell. In some embodiments, in combination is in combination with the composition.

In some embodiments, the instructions indicate the channel is for use with the receptor. In some embodiments, the labels indicate the channel is for use with the receptor. In some embodiments, the instructions indicate the receptor is for use with the channel. In some embodiments, the labels indicate the receptor is for use with the channel.

By another aspect, there is provided a method of depolarizing a cardiac cell, the method comprising expressing in the cardiac cell a PSAM4-5HT3 fusion protein, thereby depolarizing a cardiac cell.

By another aspect, there is provided a method of treating or preventing a cardiac disease or condition, the method comprising administering to the subject a PSAM4-5HT3 fusion protein, thereby treating or preventing a cardiac disease or condition. By another aspect, there is provided a method of treating or preventing a cardiac disease or condition, the method comprising administering to the subject a nucleic acid molecule encoding a PSAM4-5HT3 fusion protein, thereby treating or preventing a cardiac disease or condition. By another aspect, there is provided a method of treating or preventing a cardiac disease or condition, the method comprising administering to the subject a cardiac cell expressing a PSAM4-5HT3 fusion protein, thereby treating or preventing a cardiac disease or condition.

By another aspect, there is provide a cardiac cell expressing a PSAM4-5HT3 fusion protein. By another aspect, there is provided a composition comprising a cardiac cell expressing a PSAM4-5HT3 fusion protein.

In some embodiments, the composition is a pharmaceutical composition. In some embodiments, the composition further comprises a pharmaceutically acceptable carrier, excipient or adjuvant. In some embodiments, the composition is for use in a method of the invention. In some embodiments, expressing is expressing protein. In some embodiments, expressing is expressing mRNA. In some embodiments, expressing is expressing in the plasma membrane. In some embodiments, expressing is surface expressing. In some embodiments, the fusion protein is expressed in the plasma membrane such that PSAM4 is extracellular and 5HT3 produces a pore through the plasma membrane.

In some embodiments, method further comprises contact the cell with a ligand. In some embodiments, method further comprises administering a ligand to the subject. In some embodiments, administering a ligand is administering a composition comprising the ligand.

In some embodiments, the composition is a pharmaceutical composition. In some embodiments, the composition is formulated for systemic administration. In some embodiments, the ligand is a ligand of PSAM4. In some embodiments, the ligand is a synthetic ligand. In some embodiments, the ligand is an artificial ligand. In some embodiments, the ligand does not bind to any naturally occurring receptor. In some embodiments, the ligand does not bind to any receptor endogenous to the cell. In some embodiments, the ligand does not bind to any receptor endogenous to the subject. In some embodiments, the ligand is Varenicline.

In some embodiments, PSAM4-5HT3 is a fusion protein of the DREADD, PSAM4, and the ion channel, 5HT3. In some embodiments, the ion is a cation. In some embodiments, the ion is chloride. It is well known in the art that 5HT3 is cation selective. The fusion protein PSAM4-5HT3 has been previously disclosed in Magnus, et al., “Ultrapotent chemogenetics for research and potential clinical applications”, Science, 2019 April 12;364 (6436), herein incorporated by reference in its entirety. In some embodiments, PSAM4-5HT3 is a high conductance variant PSAM4-5HT3-HC. In some embodiments, PSAM4-5HT3 is encoded by a nucleotide sequence comprising atgcgctgttctccaggcggcgtgtggctcgccctggctgcttcccttctgcacgttagcctgcagggtgagttccagcgcaaactg tataaggagcttgttaagaattataaccccctggagcggccggtcgcaaatgattcccagccactgacagtgtacttcagcctctcct tgctgcagatcatggacgtggatgaaaagaaccaggtgctgaccactaatatttggttgcagatgtcctggaccgatcactacttgc agtggaatgtgagcgaatacccaggtgtaaagactgtaagattccctgacggccaaatctggaaaccagatatcctgctgtacaac agcgcagacgaaaggtttgatgcaacatttcacaccaacgtgggagtcaattcttcaggccactgcctgtacctgccccctggaatc ttcaagtcctcatgctatatcgacgtccgctggtttcccttcgacgtccagcactgcaaactcaaattcgggagctggagctacggcg gatggagcctggatctgcaaatgcaggaggctgacatctctggttacatcccgaatggggagtgggaccttgtgggaatccccgg taaaagaagcgagcgattttatgaatgctgcaaggaacccttccctgacgtaacattcacagttatcatcagaagaaggccattgttc tacgccgttagtttgttgctccccagtatttttctcatggtcgtggacatcgtgggattttgtctcccacctgatagcggggagagggtc tcctttaagattaccttgttgctcggctattctgtatttctgatcategtgtccgatacccttcctgccacaatcggcactccgctgatagg agtgtatttcgtcgtgtgtatggcactcctggtgataagtctggcggaaactatcttcattgtacggctggtacataagcaggacctgc aaagacccgtgccagactggttgcgacaccttgtgctggacagaattgcatggattctgtgtcttggcgagcaacctatggcccac cggccacctgcaacctttcaagccaacaagacagacgattgtagtgggtctgatctgttgcctgctatggggaatcactgctcccat gttgggggaccacaagatttggaaaagaccccacgggggggggatcaccccttcctcctccccgagaagcctctctcgctgtcc gggggctgctccaggaactgtcaagcatccgacattttctggagaagcaggacgagatggacgaagtcgctgccgactggctgc gagtgggctacgtccttgacaggctgctgtttcggatctacttgctggcggtgctggcttattccattactctggtgacactctggtcca tatggcactacagt (SEQ ID NO: 11). In some embodiments, PSAM4-5HT3 is encoded by a nucleotide sequence consisting of SEQ ID NO: 11. In some embodiments, PSAM4-5HT3 comprises the amino acid sequence MRCSPGGVWLALAASLLHVSLQGEFQRKLYKELVKNYNPLERPVANDSQPLTVY FSLSLLQIMDVDEKNQVLTTNIWLQMSWTDHYLQWNVSEYPGVKTVRFPDGQIW KPDILLYNSADERFDATFHTNVGVNSSGHCLYLPPGIFKSSCYIDVRWFPFDVQHC KLKFGSWSYGGWSLDLQMQEADISGYIPNGEWDLVGIPGKRSERFYECCKEPFPD VTFTVIIRRRPLFYAVSLLLPSIFLMVVDIVGFCLPPDSGERVSFKITLLLGYSVFLIIV SDTLPATIGTPLIGVYFVVCMALLVISLAETIFIVRLVHKQDLQRPVPDWLRHLVLD RIAWILCLGEQPMAHRPPATFQANKTDDCSGSDLLPAMGNHCSHVGGPQDLEKTP RGRGSPLPPPREASLA VRGLLQELSSIRHFLEKQDEMDEVAADWLRVGYVLDRLL FRIYLLAVLAYSITLVTLWSIWHYS (SEQ ID NO: 12). In some embodiments, PSAM4-5HT3 consists of SEQ ID NO: 12.

In some embodiments, a low dose of the ligand causes increased electrical activity in the cardiac cell. In some embodiments, the treating comprises increasing electrical activity in a cardiac cell. In some embodiments, the cardiac cell is a diseased cardiac cell. In some embodiments, the cardiac cell is a dysfunctional cardiac cell. In some embodiments, the cardiac cell is the cardiac cell expressing PSAM4-5HT3. In some embodiments, a low dose of the ligand causes increased electrical activity of cardiac cells adjacent to the cardiac cell expressing the PSAM4-5HT3. In some embodiments, the treating comprises increasing electrical activity in cardiac cells adjacent to a cardiac cell expressing PSAM4-5HT3. In some embodiments, a high dose of the ligand causes limiting of electrical activity in the cardiac cell. In some embodiments, a high dose of the ligand causes silencing of electrical activity in the cardiac cell. In some embodiments, treating comprises silencing electrical activity in the cardiac cell. In some embodiments, a high dose of the ligand causes silencing of electrical activity in cells adjacent to the cells expressing PSAM4-5HT3. In some embodiments, treating comprises silencing electrical activity in cells adjacent to the cells expressing PSAM4-5HT3. In some embodiments, the treating comprises cessation of arrythmia. In some embodiments, the treating comprises cessation of tachycardia.

In some embodiments, a low dose is a subclinical dose. In some embodiments, a high dose is a clinical dose. In some embodiments, a high dose is at least a clinical dose. In some embodiments, a low dose is a dose below a clinical dose. In some embodiments, high dose is a dose above a clinical dose. In some embodiments, a clinical dose is the minimum effective dose (MED). In some embodiments, a clinical dose is 0.5 mg. In some embodiments, a clinical dose of Varenicline is 0.5 mg. In some embodiments, a low dose is a dose below 0.5 mg. In some embodiments, a high dose is a dose of 0.5 mg or higher. In some embodiments, a high dose is a dose above 0.5 mg.

In some embodiments, the cardiac disease or condition is arrythmia. In some embodiments, the cardiac disease or condition is tachy-arrhythmia. In some embodiments, the cardiac disease or condition is brady-arrhythmia. In some embodiments, the cardiac disease or condition is bradycardia. In some embodiments, the cardiac disease or condition is tachycardia. some embodiments, the cardiac disease or condition is selected from arrythmia, tachy-arrhythmia, brady-arrhythmia, bradycardia and tachycardia.

As used herein, the term “about” when combined with a value refers to plus and minus 10% of the reference value. For example, a length of about 1000 nanometers (nm) refers to a length of 1000 nm+-100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Wherever embodiments are described with the language “comprising,” otherwise analogous embodiments described in terms of “consisting of” and/or “consisting essentially of” are included.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshncy, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategics for Protein Purification and Characterization-A Laboratory Course Manual” CSHL Press (1996); all of which are incorporated by reference. Other general references are provided throughout this document.

### Example 1: PSAM4-5HT3 Expression in Cardiomyocytes

The synthetic chemogenetic channel PSAM4-5HT3-HC was created by fusing a mutated α7-nicotinic acetylcholine receptor (PSAM4, engineered to bind the anti-smoking drug Varenicline at sub-therapeutic concentrations) with the ion pore-domain (IPD) of the serotonin receptor 3A (5HT3) with additional mutations made for high conductance (HC) (SEQ ID NO: 12). This molecule was disclosed in Magnus, et al., “Ultrapotent chemogenetics for research and potential clinical applications”, Science, 2019 April 12;364 (6436), hercin incorporated by reference in its entirety. This molecule has only ever been used in neurons; however, it was hypothesized that it could also be employed in other electrically active cells. Specifically, it was hypothesized that expressing this chemogenetic channel in cardiomyocytes would permit dose-dependent modulation of their excitable properties; with weak depolarizing currents leading to increased automaticity, and larger currents causing depolarization-induced electrical silencing.

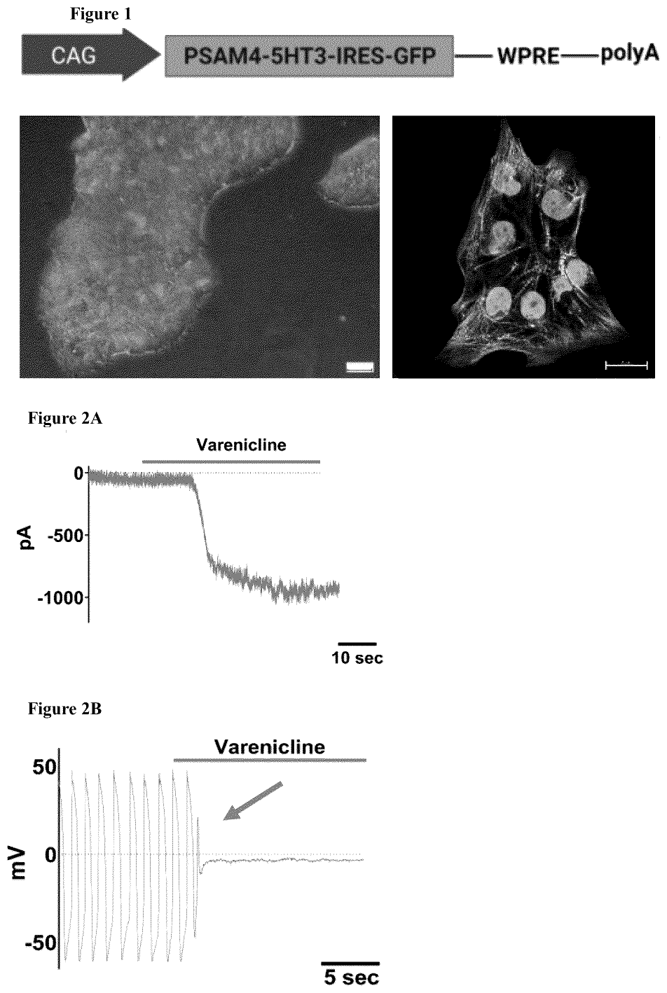

To test these hypotheses, healthy-donor human induced pluripotent stem cells (hiPSCs) were transfected with a non-viral, randomly integrating, transposon to create a stably engineered line constitutively expressing the PSAM4-5HT3-HC channel together with cGFP (FIG. 1). Whole-genome sequencing confirmed that no exons were affected. Next, the genetically-modified hiPSCs were differentiated into cardiomyocytes (hiPSC-CMs), which maintained robust expression of the synthetic channel (FIG. 1, bottom-right).

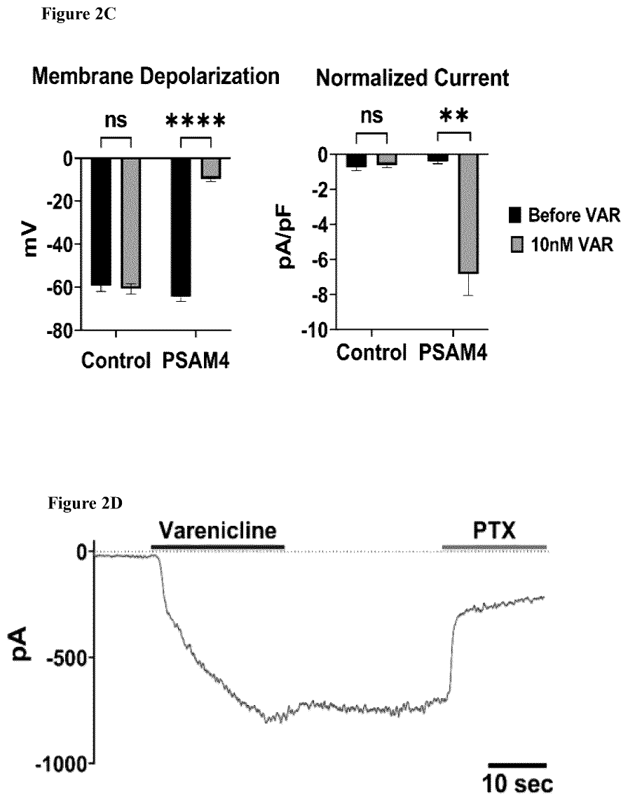

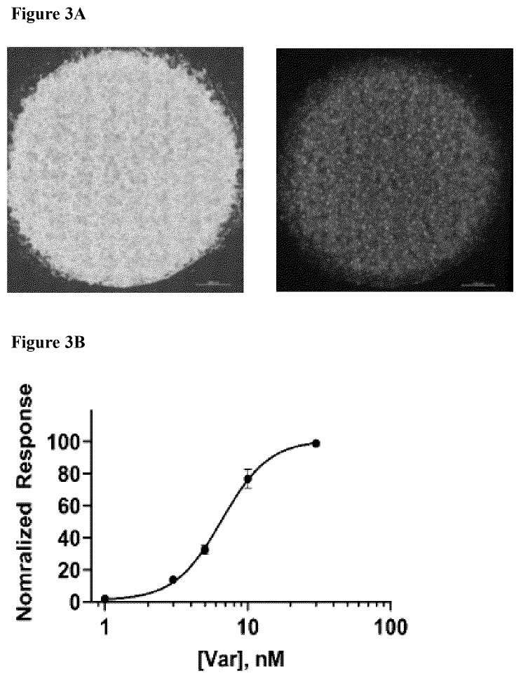

Voltage-clamp analysis of the PSAM4-5HT3-HC-expressing hiPSC-CMs revealed robust Varenicline-induced depolarizing currents (FIG. 2A), even at low concentrations (1 nM). At higher doses (10 nM), spontaneous generation of action potentials (Aps) ceased (FIG. 2B). This suppression of excitability likely results from the significant membrane diastolic depolarization (−64±7 mV to −10±4 mV; p<0.0001; FIG. 2C) due to the large Varenicline-induced inward current (−6.8±3.2 pA/pF; p<0.01; FIG. 2C). Application of picrotoxin, a non-competitive partial antagonist of 5HT3-IPD, antagonized Varenicline's effect, suppressing the depolarizing current (FIG. 2D). No response to Varenicline was observed in control unmodified hiPSC-CMs (FIG. 2C).

### Example 2: PSAM4-5HT3 Expression in Cardiac Tissue

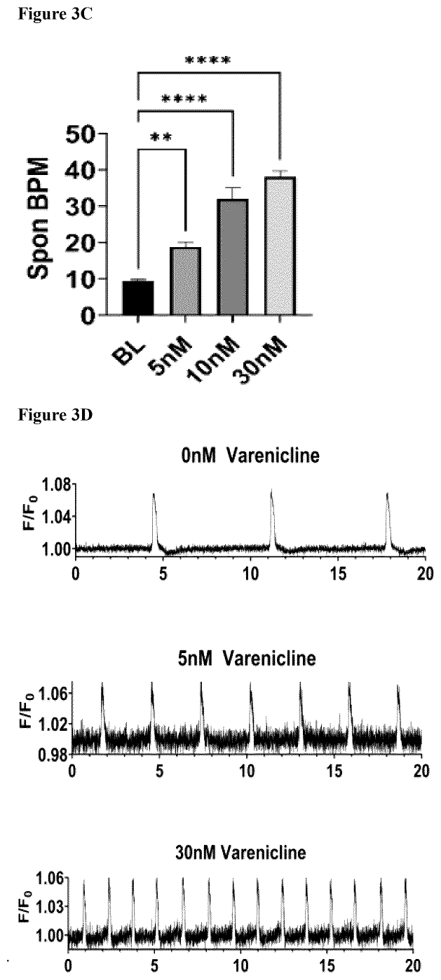

To assess the translational potential of these findings, a hiPSC-derived cardiac cell-sheet (hiPSC-CCS) tissue model generated from PSAM4-5HT3-hiPSC-CMs was utilized (FIG. 3A). When using Varenicline concentrations between 5-30 nM, optical mapping showed a dose-dependent increase in the cultures' activation frequency (FIG. 3B-3D). This augmented automaticity was the result of Varenicline-induced depolarizing currents in pacemaker cells driving activity of the cultures. In contrast, no chronotropic effects were observed at any dose of Varenicline in control, non-transfected, hiPSC-CCSs.

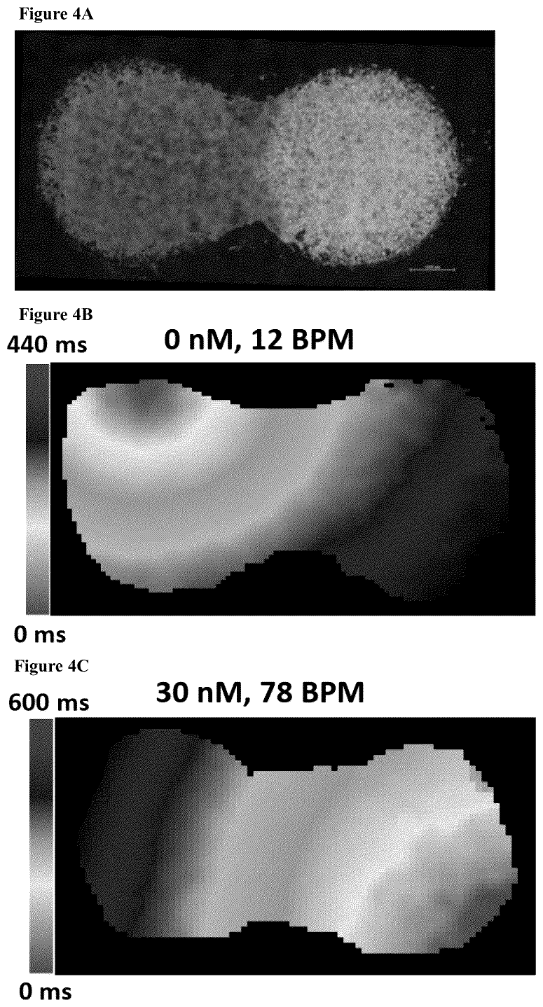

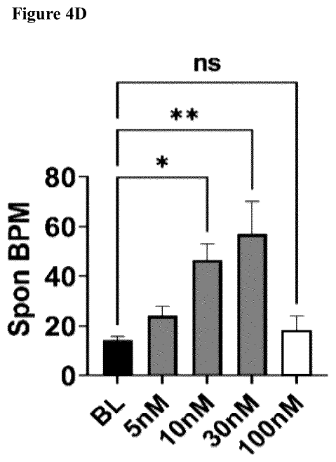

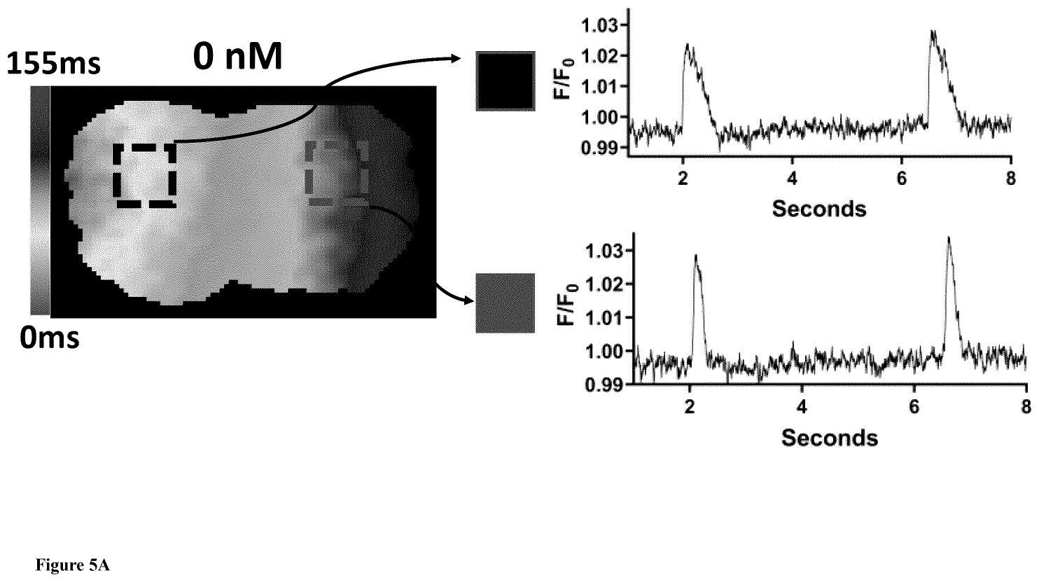

We next created a co-culture model where the PSAM4-5HT3-cGFP-hiPSC-CMs were seeded beside control hiPSC-CMs (FIG. 4A). Notice that at baseline (FIG. 4B), spontaneous activity originated (red) from the control tissue, whereas exposure to Varenicline both shifted the pacemaker area to the PSAM4-5HT3-HC tissue (FIG. 4C) and augmented automaticity in a dose-dependent manner (FIG. 4D).

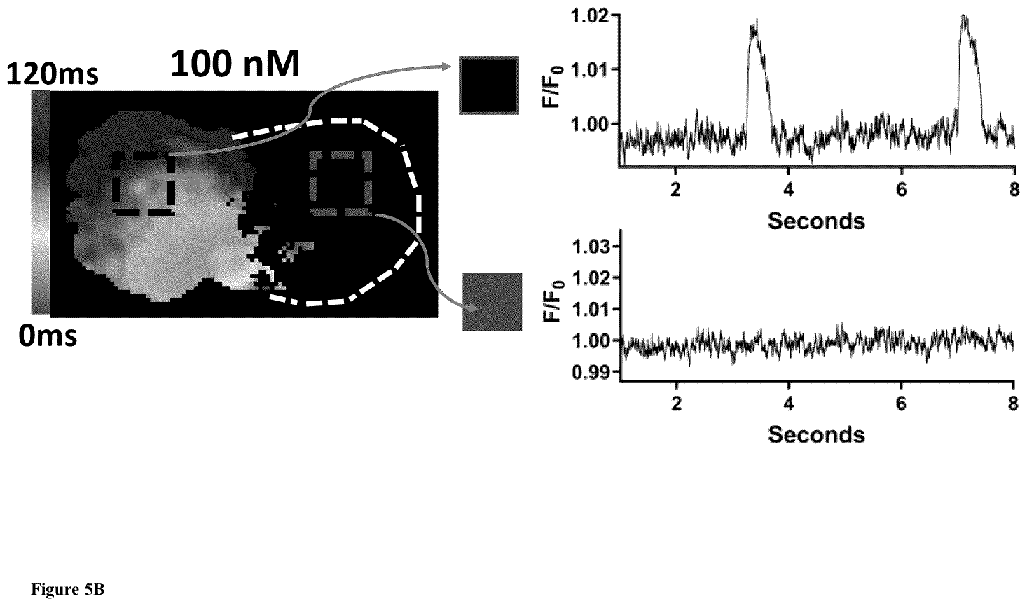

Next, it was tested whether this chemogenetic approach could suppress tissue automaticity and excitability when using higher concentrations of Varenicline (100 nM), as it did in single cells, albeit at lower concentration. This dosing discrepancy probably stems from the more limited drug diffusion and the presence of stronger electrotonic coupling (attenuating Varenicline-induced depolarization) at the tissue level. Consequentially, 100 nM Varenicline significantly slowed spontaneous activity of the co-cultures (FIG. 4D). Moreover, in some specimens, in contrast to baseline activity (FIG. 5A), complete silencing of electrical activity in the PSAM4-5HT3-hiPSC-CMs area following application of 100 nM Varenicline was observed, whereas the region containing the non-transfected cells retained its excitable properties (FIG. 5B).

### Example 3: PSAM4-5HT3 Expression in in vivo Hearts

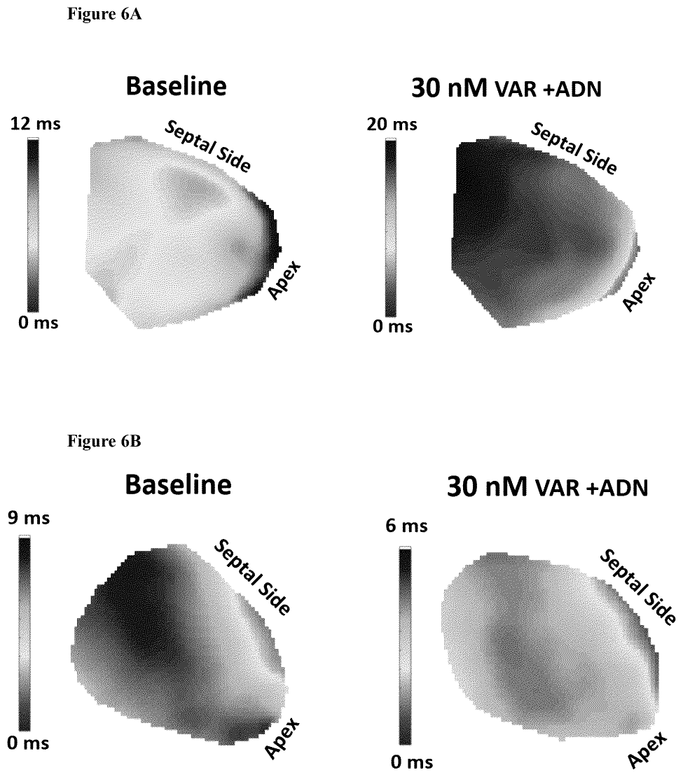

Finally, chemogenetic hiPSC-CMs were transplanted into rat hearts. After 7-10 days, hearts were harvested, perfused ex-vivo using a custom-made Langendorff apparatus and optically mapped. Bradycardia was induced using adenosine. Upon exposure to Varenicline (30 nM) ectopic ventricular activity developed in four out of five hearts studied, whose origin was optically mapped to the area of cell transplantation at the apex (FIG. 6A, right-panel). This shift in the activation pattern did not occur in four control hearts (FIG. 6B).

### Example 4: SthK-Rm3D System

A limitation of inactivation by depolarization, as shown above, is that long term depolarization of cells is not physiologically acceptable, and may lead to cell death over time, thus, such a solution is relevant primarily for acute, short, electrical inactivation. Conversely, the drawback of a system based on chloride anion channels such as PSAM4-GlyR is that many cells are not hyperpolarized by chloride channel activation but rather depolarized. This is dependent on intracellular/extracellular chloride concentrations and the resting membrane potential (together determining the Nernst potential). Another option would be the use of an existing DREADD, G-coupled receptor Hm4di, which has been shown to electrically inhibit neurons. This inhibition is the product of endogenous G-coupled inwardly rectifying potassium channels (GIRKs) and through a direct inhibition of synaptic transmission. Interestingly, this DREADD only leads to very modest hyperpolarization, and most of the inhibitory capacity is due to the synaptic inhibition. Thus, this DREADD is insufficient for cardiac electrical silencing, and only leads to modest hyperpolarization, altering, but not eliminating, electrical excitability in cardiomyocytes. In-vitro experiments with both a chloride PSAM/PSEM4 (PSAM4-GlyR.) and with the hm4di DREADD were performed. The two molecules were expressed in iPSCs differentiated to cardiomyocytes and then cells were then treated with synthetic ligand. It was observed that neither is sufficient to completely inhibit electrical activity in cardiomyocytes in-vitro.

To overcome the insufficiency of these methods, a cyclic adenosine monophosphate (cAMP) producing DREADD, Rm3D, was selected for further study. It was hypothesized that combination of this DREADD with a cAMP-gated potassium channel (as opposed to a chloride channel) would allow for significant membrane hyperpolarization, sufficient to completely inhibit electrical activity in cardiomyocytes. The potassium channel selected was the SthK channel. Rm3D is an excitatory DREADD derived from the M3 muscarinic receptor, and it is chemogenetically activated by either clozapine-N-oxide (CNO) or DREADD agonist 21/compound 21 (C21).

In order to express both the DREADD and the channel in the membrane of cardiomyocytes a vector encoding a single transcript was generated. The open reading frame for SthK was cloned downstream of the chicken B-actin promoter. Instead of a stop codon, at the end of SthK a P2A cleavable peptide was encoded followed by the Rm3D receptor in frame. The result is a reading frame (SEQ ID NO: 4) encoding an 874 amino acid construct (SEQ ID NO: 8) that is co-translationally cleaved near the end of the P2A peptide to produce separate SthK and Rm3D proteins from a single transcript.

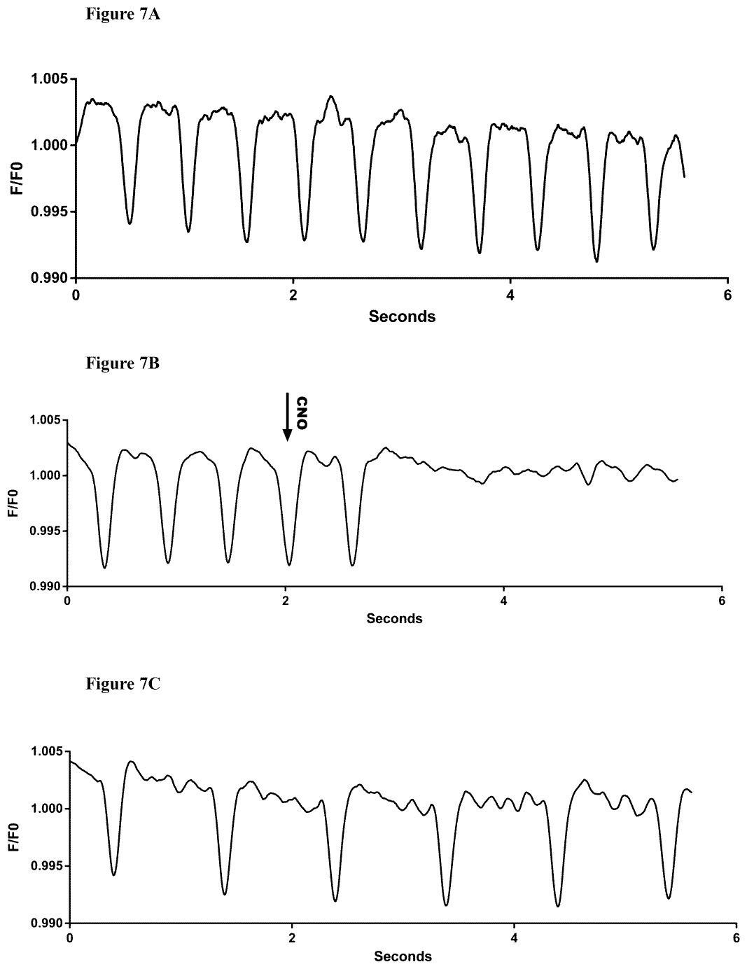

The construct was transfected into human induced pluripotent stem cells (hiPSCs) which were observed to spontaneously contract after differentiation to cardiomyocytes (iPSC-CM). Cardiac cell sheets (CCSs) were generated as a 2D tissue model from the transfected iPSC-CMs and the CCSs were loaded with an optical voltage-dependent dye and mapped using a high-speed EMCCD camera. The CCSs demonstrated robust spontaneous beating and responded well to electrical pacing. Reentrant arrhythmias were induced by transient tachypacing, and the resultant arrhythmias were stable within the tissue (FIG. 7A). Very low doses of CNO (2-5 nM) resulted in rapid inactivation (silencing) of the electrical activity of the CCS, and no conduction or activity was visible in the optical maps (FIG. 7B). This was seen as an inability to excite the cells even with significant electrical stimulation. These very low doses were sufficient to completely terminate existing arrhythmias. Furthermore, it was shown that a single washout of the drug led to rapid recovery of electrical excitability by electrical pacing (within 30-90 seconds of washout) and the spontaneous resumption of a normal rhythm (sinus) within 2-5 minutes, as would be expected after defibrillation/cardioversion (FIG. 7C). Thus, the arrythmias were successfully terminated upon application of CNO, and normal electrical activity returned almost immediately after washout.

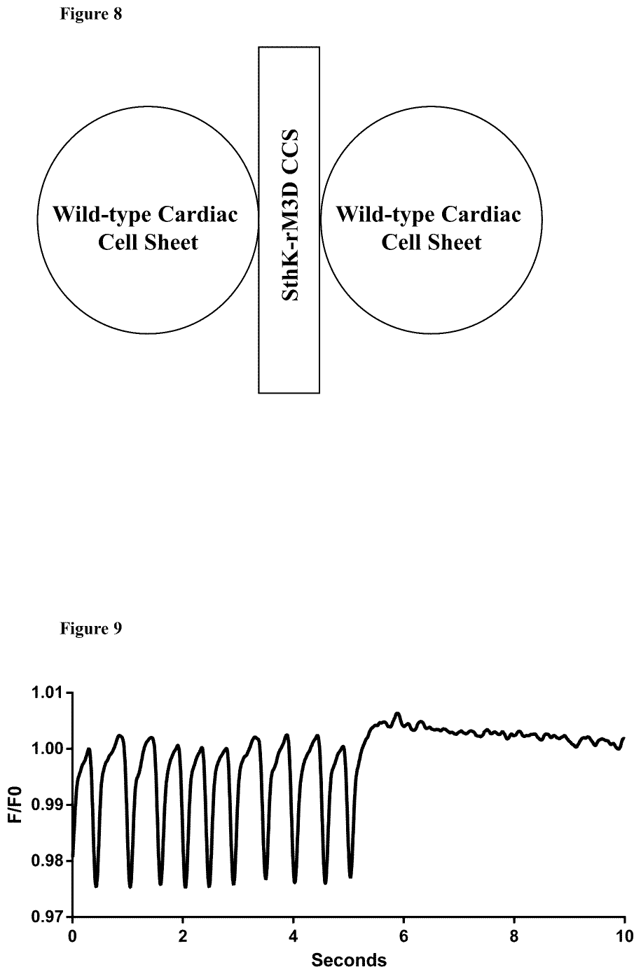

To further illustrate the potential of this technology a coculture model of wild type CMs and CMs engineered to express the compound channel was generated (FIG. 8). In this model, only the CMs in the CCS between the two wild-type CCSs express the compound channel that is responsive to CNO. Reentrant arrhythmias were induced that spanned the whole model. Upon application of CNO (5 nM) the arrhythmia could no longer cross the engineered cells which became unexcitable due to the chemogenetic hyperpolarization. As such, the arrhythmia became relegated to a single side of the model. That is an arrhythmia with a core in one CCS (for example the right one) become relegate to just that CCS upon application of CNO, while the other (left) CCS remained unaffected. It was further shown that the left CCS could be electrically paced, and conduction was successful until the wave front reaches the unexcitable engineered CMs. The phenomenon was also demonstrated in reverse in the same model. Thus, it is clear that native CMs are unaffected by CNO, but the genetically engineered CMs can be robustly ‘silenced’ by the application of CNO at very low concentrations (2-5 nM). Further, arrhythmias are succinctly terminated as a result of this silencing. Upon washout normal electrical activity resumed in the engineered CMs.

To further test the feasibility of this technology in an animal, a model was generated in which only some of the cells of the CCS contained the construct. It is likely that during delivery of the construct to cardiac cells not every cell of the tissue will receive and express the construct of the invention. In fact, therapies that rely on viral infection are often hampered by poor transfer of the therapeutic molecule. CCSs were generated in which engineered cardiomyocytes were mixed with isogenic control cardiomyocytes that do not express the construct. CCSs with ratios from 5% engineered cells and 95% control cells to 100% engineered cells to 0% control cells were generated. The engineered cells were evenly spread throughout the CCS creating a homogenous sheet. Rotor-like arrhythmias within the monolayer were induced by electrical tachypacing and the channel was activated by the administration of drug. Optical mapping showed that the arrythmias were rapidly terminated (within ˜1 minute) in CCSs containing as little as 10% of the cells receiving the therapeutic construct of the invention. An optical trace from a CCS containing 10% engineered cells showing the termination of the arrythmia is provided in FIG. 9. Even in constructs with only 5% engineered cells, the addition of the drug was sufficient to terminate the arrythmia, though the effect was not as rapid. This result demonstrates one of the important unexpected benefits of the use of the chemogenetically gated channel of the invention: therapeutic effect within the heart can be achieved even with a low level of construct delivery to the tissue.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

What is claimed is:

1. A nucleic acid molecule comprising a first sequence encoding a cyclic nucleotide gated potassium channel or a functional fragment thereof and a second sequence encoding a chemogenetically activatable cyclic nucleotide generating receptor or a functional fragment thereof.

2. The nucleic acid molecule of claim 1, wherein said cyclic nucleotide gated potassium channel is SthK or wherein a sequence encoding said cyclic nucleotide gated potassium channel comprises SEQ ID NO: 1 or a sequence with at least 85% identity to SEQ ID NO: 1 that is an ion channel that is responsive to potassium

3. (canceled)

4. (canceled)

5. The nucleic acid molecule of claim 1, wherein at least one of:
a. said chemogenetically activatable cyclic nucleotide generating receptor is a DREADD;
b. said chemogenetically activatable cyclic nucleotide generating receptor is an excitatory DREADD;
c. said chemogenetically activatable cyclic nucleotide generating receptor is a DREADD derived from human M3 muscarinic receptor (hM3);
d. said chemogenetically activatable cyclic nucleotide generating receptor is rM3D; and
e. said chemogenetically activatable cyclic nucleotide generating receptor is encoded by SEQ ID NO: 2 or a sequence with at least 85% identity thereto

6. (canceled)

7. (canceled)

8. (canceled)

9. (canceled)

10. The nucleic acid molecule of claim 1, wherein said first sequence and said second sequence are in the same open reading frame.

11. (canceled)

12. The nucleic acid molecule of claim 10, wherein at least one of:
a. said nucleic acid molecule is a DNA molecule and wherein a single open reading frame encodes an mRNA translatable to said cyclic nucleotide gated potassium channel and said chemogenetically activatable cAMP generating receptor;
b. said nucleic acid molecule comprises a third sequence encoding a linker peptide between said first sequence and said second sequence;
c. said nucleic acid molecule comprises a third sequence encoding a cleavable linker peptide between said first sequence and said second sequence;
d. said nucleic acid molecule comprises a third sequence encoding a P2A peptide between said first sequence and said second sequence; and
e. said nucleic acid molecule comprises a third sequence encoding SEO ID NO: 3 between said first sequence and said second sequence.

13. (canceled)

14. (canceled)

15. (canceled)

16. (canceled)

17. The nucleic acid molecule of claim 1,
a. encoding a protein comprising or consisting of SEQ ID NO: 8;
b. comprising SEQ ID NO: 4; or
c. both

18. (canceled)

19. An expression vector comprising a nucleic acid molecule of claim 1 operatively linked to at least one transcriptional regulatory element.

20. The expression vector of claim 19, wherein
a. said at least one transcriptional regulatory element is a promoter;
b. said at least one transcriptional regulatory element is a constitutively active promoter;
c. said at least one transcriptional regulatory element is a promoter specifically active in cardiac cells; or
d. said at least one transcriptional regulatory element comprises a cardiac cell specific enhancer.

21. (canceled)

22. (canceled)

23. A fusion protein comprising a cyclic nucleotide gated potassium channel or a functional fragment thereof and a chemogenetically activatable CAMP generating receptor or a functional fragment thereof.

24. The fusion protein of claim 23, wherein
a. said cyclic nucleotide gated potassium channel is SthK;
b. said chemogenetically activatable cAMP generating receptor is rM3D;
c. said cyclic nucleotide gated potassium channel comprises SEQ ID NO: 5 or a functional fragment thereof or sequence with at least 85% identity thereto:
d. said chemogenetically activatable CAMP generating receptor comprises SEQ ID NO: 6 or a functional fragment thereof or sequence with at least 85% identity thereto; or
e. a combination thereof.

25. (canceled)

26. The fusion protein of claim 23, encoded by a nucleic acid molecule of claim 1.

27. The fusion protein of claim 23, comprising the amino acid sequence provided in SEQ ID NO: 28.

28. A cell comprising a nucleic acid molecule of claim 1, optionally wherein said cell is a cardiac cell, optionally wherein said cardiac cell is a cardiomyocyte

29. (canceled)

30. A pharmaceutical composition comprising a nucleic acid molecule of claim 1 and a pharmaceutically acceptable carrier, excipient or adjuvant, optionally being formulated for administration to a subject.

31. (canceled)

32. A method of hyperpolarizing a cell, the method comprising expressing in said cell a nucleic acid molecule of claim 1, and contacting said cell with a ligand of said chemogenetically activatable cyclic nucleotide generating receptor, optionally wherein said ligand is clozapine-N-oxide (CNO) or DREADD agonist 21/compound 21 (C21), thereby hyperpolarizing a cell.

33. (canceled)

34. (canceled)

35. A method of treating or preventing a disease or condition in a subject in need thereof, the method comprising administering to said subject a pharmaceutical composition of claim 30 and further administering a ligand of said chemogenetically activatable cyclic nucleotide generating receptor, optionally wherein said ligand is CNO or C21, thereby treating a cardiac disease or condition.

36. (canceled)

37. The method of claim 35, wherein
a. said disease or condition is characterized by electrical disfunction in a disease tissue or cell;
b. said disease or condition is a cardiac disease or condition and is characterized by electrical disfunction in a disease tissue or cell;
c. said disease or condition is a cardiac disease or condition selected from: arrhythmia, tachy-arrhythmia, brady-arrhythmia, bradycardia and tachycardia
d. said disease or condition is a neurological disease or condition caused by hyperactivity of a neuron;
e. said disease or condition is a neurological disease or condition selected from: epilepsy Parkinson's and parkinsonian syndromes. essential tremor restless leg syndrome tinnitus pain and phantom sensations. Alzheimer's disease and neuropathy;
f. said disease or condition is a smooth muscle disease or condition selected from benign prostatic hyperplasia (BPH), hypertension, erectile dysfunction, coronary artery disease, pathologies of the stomach and intestines leading to lack of motility or hyper motility achalasia, gastroesophageal reflux disease (GERD), urinary incontinence and urinary retention; or
g. said disease or condition is a striated muscle disease or condition requiring muscle relaxation.

38. (canceled)

39. (canceled)

40. (canceled)

41. (canceled)

42. (canceled)

43. (canceled)

44. (canceled)

45. (canceled)

46. A kit comprising:
a. a nucleic acid molecule of claim 1; and
b. a ligand of said chemogenetically activatable cyclic nucleotide generating receptor, optionally wherein said ligand is CNO or C21.

47. (canceled)

48. (canceled)

49. (canceled)

50. A method of treating or preventing a cardiac disease or condition in a subject in need thereof the method comprising administering to said subject a PSAM4-5HT3 fusion protein, a nucleic acid molecule encoding said PSAM4-5HT3 fusion protein or a pharmaceutical composition comprising said PSAM4-5HT3 fusion protein or said nucleic acid molecule encoding said PSAM4-5HT3 fusion protein and a pharmaceutically acceptable carrier, excipient or adjuvant, and administering to said subject a ligand of PSAM4, optionally wherein PSAM4-5HT3 comprises SEQ ID NO: 12, said ligand is Varenicline or both, thereby treating a cardiac disease or condition.

51. (canceled)

52. The method of claim 50, wherein
a. a low dose of said ligand causes increased electrical activity of said cardiac cell or adjacent cardiac cells and a high dose of said ligand causes complete silencing of electrical activity in said cardiac cell or adjacent cardiac cells;
b. a subclinical dose of said ligand causes increased electrical activity of said cardiac cell or adjacent cardiac cells and a clinical dose or higher of said ligand causes complete silencing of electrical activity in said cardiac cell or adjacent cardiac cells;
c. a dose of Varenicline below 0.5 mg causes increased electrical activity of said cardiac cell or adjacent cardiac cells and a dose of Varenicline of 0.5 mg or higher causes complete silencing of electrical activity in said cardiac cell or adjacent cardiac cells; or
d. said disease or condition is selected from arrythmia, tachy-arrhythmia, brady-arrhythmia, bradycardia and tachycardia.

53. (canceled)

54. (canceled)

55. (canceled)

56. (canceled)


SEQUENCE LISTING (28 sequences)

SEQ ID NO: 1 - DNA, 1329 bp, Escherichia coli
  source 1..1329: mol_type = genomic DNA
atgaaaagctccgccttctcccaccccacctacaccctggtctggaaagtcggcattctggctgtcactctgtattacgctattcgaatcccactgaccctggtgttcccctctctgtttagtcccctgctgcctctggatatcctggccagtctggctctgatcgcagacattcctctggatttcgcctttgagtcacgaaagacaagcggcaggaaaccaactctgctggctcctagccgactgccagatctgctggccgctctgccactggacctgctggtgttcgccctgcacctgccatcacccctgagcctgctgtccctggtgaggctgctgaagctgatctccgtccagaggtctgctacaagaatcctgtcttacagaattaacccagcactgctgcggctgctgagtctggtgggattcatcctgctggcagcccatgggattgcctgcggctggatgtcactgcagccacctagcgagtccccagcaggaaccagatacctgagcgccttctactggacaatcaccacactgactaccatcggctacggagatattaccccatccacacccattcagaccgtgtacaccatcgtcattgagctgctgggagctgcaatgtatggactggtcatcgggaatattgcatctctggtcagtaagctggacgccgctaaactgctgcaccgagagaggatggaacgggtgacagctttcctgagttacaagaaaatctcacctgagctgcagaggagaattctggaatactttgattatctgtgggagactcggcgcgggtatgaggaacgcgaggtgctgaaggaactgcctcacccactgcgactggctgtcgcaatggaaatccatggcgacgtgattgagaaggtcccactgttcaaaggggccggcgaagactttatccgcgatatcattctgcatctggagcccgtgatctacggacctggggaatatatcattagggctggcgagctgggcagcgatgtctactttatcaacagaggcagcgtggaggtcctgtccgcagacgaaaagacccggtatgccatcctgtctgagggccagttctttggagaaatggcactgattctgcgagcaccacgaacagctactgtgagagcacggactttctgtgacctgtacagactggataaagaaacctttgacagaatcctgtctcgctatcctgagattgcagcccagattcaggaactggctgtgcggaggaaagaagaactggaaggggggacatcacggcggggaaccggtcccgggcttaaggagctcgcatgcggaagcgga

SEQ ID NO: 2 - DNA, 1236 bp, synthetic construct
  source 1..1236: mol_type = other DNA
atgaccttgcacagtaacagtacaacctcgcctttgtttcccaacatcagctcttcctgggtgcacagtccctcggaggcagggctgcccttggggacagtcactcagttgggcagctacaacatttcacaagaaactgggaatttctcctcaaacgacacctccagcgaccctctcgggggtcacaccatctggcaagtggtcttcattgccttcttaactggcttcctggcattggtgaccatcattggcaacatccttgtcattgtggccttcaaggtcaacaaacagctgaagacagtcaacaactacttcctcttaagcctggcctgtgcagacctgatcatcggggtcatttccatgaacctgttcactacctacatcattatgaaccgttgggcactggggaacttagcctgcgacctctggctctccattgactgtgtggccagcaatgcctctgtcatgaatctgctggtcatcagctttgacaggtacttttccatcacttctccattccgctaccagagcctgatgaccagggctcgagctggtgtgatgattggtctggcttgggtcatctcctttgtcctatgggctcctgccatcttgttctggcaatactttgtagggaagagaactgtgcccccaggagaatgtttcattcagtttctgagtgagcccaccatcaccttcggcacggcgatcgctggcttttacatgcctgtcaccatcatgactattttatactggcgggtgtaccgggaggccaaggagcagatcaggaagatcgaccgctgcgagggccggttctatggcagccaggagcagccgcagccacccccgctcccccaacaccagcccatcctcggcaacggccgtgccagcaagaggaagacgtcccgtgtcatggccatgagggaacacaaagctctgcagacgctcagtgccatcttgctggccttcatcatcacgtggaccccctacaacatcatggtcctggtgaacaccttctgtgacagctgcatacccaaaacctattggaatctgggctactggctgtgctatatcaacagcaccgtgaaccctgtgtgctatgccctgtgcaacaaaacattcagaaccaccttcaagacgctcctcttgtgccagtgtgacaaaaggaagaggcgcaaacagcagtaccagcagagacagtcggtcatttttcacaagcgagtgccggagcaggccttg

SEQ ID NO: 3 - DNA, 57 bp, synthetic construct
  source 1..57: mol_type = other DNA
gccacgaacttctctctgttaaagcaagcaggagacgtggaagaaaaccccggtcct

SEQ ID NO: 4 - DNA, 2622 bp, synthetic construct
  source 1..2622: mol_type = other DNA
atgaaaagctccgccttctcccaccccacctacaccctggtctggaaagtcggcattctggctgtcactctgtattacgctattcgaatcccactgaccctggtgttcccctctctgtttagtcccctgctgcctctggatatcctggccagtctggctctgatcgcagacattcctctggatttcgcctttgagtcacgaaagacaagcggcaggaaaccaactctgctggctcctagccgactgccagatctgctggccgctctgccactggacctgctggtgttcgccctgcacctgccatcacccctgagcctgctgtccctggtgaggctgctgaagctgatctccgtccagaggtctgctacaagaatcctgtcttacagaattaacccagcactgctgcggctgctgagtctggtgggattcatcctgctggcagcccatgggattgcctgcggctggatgtcactgcagccacctagcgagtccccagcaggaaccagatacctgagcgccttctactggacaatcaccacactgactaccatcggctacggagatattaccccatccacacccattcagaccgtgtacaccatcgtcattgagctgctgggagctgcaatgtatggactggtcatcgggaatattgcatctctggtcagtaagctggacgccgctaaactgctgcaccgagagaggatggaacgggtgacagctttcctgagttacaagaaaatctcacctgagctgcagaggagaattctggaatactttgattatctgtgggagactcggcgcgggtatgaggaacgcgaggtgctgaaggaactgcctcacccactgcgactggctgtcgcaatggaaatccatggcgacgtgattgagaaggtcccactgttcaaaggggccggcgaagactttatccgcgatatcattctgcatctggagcccgtgatctacggacctggggaatatatcattagggctggcgagctgggcagcgatgtctactttatcaacagaggcagcgtggaggtcctgtccgcagacgaaaagacccggtatgccatcctgtctgagggccagttctttggagaaatggcactgattctgcgagcaccacgaacagctactgtgagagcacggactttctgtgacctgtacagactggataaagaaacctttgacagaatcctgtctcgctatcctgagattgcagcccagattcaggaactggctgtgcggaggaaagaagaactggaaggggggacatcacggcggggaaccggtcccgggcttaaggagctcgcatgcggaagcggagccacgaacttctctctgttaaagcaagcaggagacgtggaagaaaaccccggtcctatgaccttgcacagtaacagtacaacctcgcctttgtttcccaacatcagctcttcctgggtgcacagtccctcggaggcagggctgcccttggggacagtcactcagttgggcagctacaacatttcacaagaaactgggaatttctcctcaaacgacacctccagcgaccctctcgggggtcacaccatctggcaagtggtcttcattgccttcttaactggcttcctggcattggtgaccatcattggcaacatccttgtcattgtggccttcaaggtcaacaaacagctgaagacagtcaacaactacttcctcttaagcctggcctgtgcagacctgatcatcggggtcatttccatgaacctgttcactacctacatcattatgaaccgttgggcactggggaacttagcctgcgacctctggctctccattgactgtgtggccagcaatgcctctgtcatgaatctgctggtcatcagctttgacaggtacttttccatcacttctccattccgctaccagagcctgatgaccagggctcgagctggtgtgatgattggtctggcttgggtcatctcctttgtcctatgggctcctgccatcttgttctggcaatactttgtagggaagagaactgtgcccccaggagaatgtttcattcagtttctgagtgagcccaccatcaccttcggcacggcgatcgctggcttttacatgcctgtcaccatcatgactattttatactggcgggtgtaccgggaggccaaggagcagatcaggaagatcgaccgctgcgagggccggttctatggcagccaggagcagccgcagccacccccgctcccccaacaccagcccatcctcggcaacggccgtgccagcaagaggaagacgtcccgtgtcatggccatgagggaacacaaagctctgcagacgctcagtgccatcttgctggccttcatcatcacgtggaccccctacaacatcatggtcctggtgaacaccttctgtgacagctgcatacccaaaacctattggaatctgggctactggctgtgctatatcaacagcaccgtgaaccctgtgtgctatgccctgtgcaacaaaacattcagaaccaccttcaagacgctcctcttgtgccagtgtgacaaaaggaagaggcgcaaacagcagtaccagcagagacagtcggtcatttttcacaagcgagtgccggagcaggccttg

SEQ ID NO: 5 - protein, 443 aa, Escherichia coli
MKSSAFSHPTYTLVWKVGILAVTLYYAIRIPLTLVFPSLFSPLLPLDILASLALIADIPLDFAFESRKTSGRKPTLLAPSRLPDLLAALPLDLLVFALHLPSPLSLLSLVRLLKLISVQRSATRILSYRINPALLRLLSLVGFILLAAHGIACGWMSLQPPSESPAGTRYLSAFYWTITTLTTIGYGDITPSTPIQTVYTIVIELLGAAMYGLVIGNIASLVSKLDAAKLLHRERMERVTAFLSYKKISPELQRRILEYFDYLWETRRGYEEREVLKELPHPLRLAVAMEIHGDVIEKVPLFKGAGEDFIRDIILHLEPVIYGPGEYIIRAGELGSDVYFINRGSVEVLSADEKTRYAILSEGQFFGEMALILRAPRTATVRARTFCDLYRLDKETFDRILSRYPEIAAQIQELAVRRKEELEGGTSRRGTGPGLKELACGSG

SEQ ID NO: 6 - protein, 412 aa, synthetic construct
MTLHSNSTTSPLFPNISSSWVHSPSEAGLPLGTVTQLGSYNISQETGNFSSNDTSSDPLGGHTIWQVVFIAFLTGFLALVTIIGNILVIVAFKVNKQLKTVNNYFLLSLACADLIIGVISMNLFTTYIIMNRWALGNLACDLWLSIDCVASNASVMNLLVISFDRYFSITSPFRYQSLMTRARAGVMIGLAWVISFVLWAPAILFWQYFVGKRTVPPGECFIQFLSEPTITFGTAIAGFYMPVTIMTILYWRVYREAKEQIRKIDRCEGRFYGSQEQPQPPPLPQHQPILGNGRASKRKTSRVMAMREHKALQTLSAILLAFIITWTPYNIMVLVNTFCDSCIPKTYWNLGYWLCYINSTVNPVCYALCNKTFRTTFKTLLLCQCDKRKRRKQQYQQRQSVIFHKRVPEQAL

SEQ ID NO: 7 - protein, 19 aa, synthetic construct
ATNFSLLKQAGDVEENPGP

SEQ ID NO: 8 - protein, 874 aa, synthetic construct
MKSSAFSHPTYTLVWKVGILAVTLYYAIRIPLTLVFPSLFSPLLPLDILASLALIADIPLDFAFESRKTSGRKPTLLAPSRLPDLLAALPLDLLVFALHLPSPLSLLSLVRLLKLISVQRSATRILSYRINPALLRLLSLVGFILLAAHGIACGWMSLQPPSESPAGTRYLSAFYWTITTLTTIGYGDITPSTPIQTVYTIVIELLGAAMYGLVIGNIASLVSKLDAAKLLHRERMERVTAFLSYKKISPELQRRILEYFDYLWETRRGYEEREVLKELPHPLRLAVAMEIHGDVIEKVPLFKGAGEDFIRDIILHLEPVIYGPGEYIIRAGELGSDVYFINRGSVEVLSADEKTRYAILSEGQFFGEMALILRAPRTATVRARTFCDLYRLDKETFDRILSRYPEIAAQIQELAVRRKEELEGGTSRRGTGPGLKELACGSGATNFSLLKQAGDVEENPGPMTLHSNSTTSPLFPNISSSWVHSPSEAGLPLGTVTQLGSYNISQETGNFSSNDTSSDPLGGHTIWQVVFIAFLTGFLALVTIIGNILVIVAFKVNKQLKTVNNYFLLSLACADLIIGVISMNLFTTYIIMNRWALGNLACDLWLSIDCVASNASVMNLLVISFDRYFSITSPFRYQSLMTRARAGVMIGLAWVISFVLWAPAILFWQYFVGKRTVPPGECFIQFLSEPTITFGTAIAGFYMPVTIMTILYWRVYREAKEQIRKIDRCEGRFYGSQEQPQPPPLPQHQPILGNGRASKRKTSRVMAMREHKALQTLSAILLAFIITWTPYNIMVLVNTFCDSCIPKTYWNLGYWLCYINSTVNPVCYALCNKTFRTTFKTLLLCQCDKRKRRKQQYQQRQSVIFHKRVPEQAL

SEQ ID NO: 9 - DNA, 1767 bp, Rattus norvegicus
  source 1..1767: mol_type = genomic DNA
atgaccttgcacagtaacagtacaacctcgcctttgtttcccaacatcagctcttcctgggtgcacagtccctcggaggcagggctgcccttggggacagtcactcagttgggcagctacaacatttcacaagaaactgggaatttctcctcaaacgacacctccagcgaccctctcgggggtcacaccatctggcaagtggtcttcattgccttcttaaccggcttcctggcattggtgaccatcattggcaacatccttgtcattgtggccttcaaggtcaacaaacagctgaagacagtcaacaactacttcctcttaagcctggcctgtgcagacctgatcatcggggtcatttccatgaacctgttcactacctacatcattatgaaccgttgggcactggggaacttagcctgcgacctctggctttccattgactatgtggccagcaatgcctctgtcatgaatctgctggtcatcagctttgacaggtacttttccatcactagaccactcacctaccgagccaaaagaacaacaaaacgagctggtgtgatgattggtctggcttgggtcatctcctttgtcctatgggctcctgccatcttgttctggcaatactttgtagggaagagaactgtgcccccaggagaatgtttcattcagtttctgagtgagcccaccatcaccttcggcacggcgatcgctgccttttacatgcctgtcaccatcatgactattttatactggaggatctataaggaaactgagaagcgtaccaaagagctggctggcctacaggcctctgggacagaagcggaggcagaaaactttgtccaccccacaggcagttctcgaagctgtagcagctatgaactgcaacagcaaggcgtgaaacgatcatccaggaggaagtacggtcgctgtcacttctggttcaccaccaagagctggaagcccagtgccgagcagatggaccaagaccacagcagcagcgacagttggaacaacaacgatgctgctgcctccctggaaaactctgcttcctccgatgaagaggacattggctcagagaccagggccatctattccattgtcctcaagcttccaggccatagctccatcctcaactctaccaagctaccgtcctcagataacctgcaggtgtccaacgaggacctggggactgtggatgtggagagaaatgctcacaagcttcaggcccagaagagcatgggtgatggtgacaactgtcagaaggatttcaccaagcttcccatccagttagagtctgccgtggacacaggcaagacctctgacaccaactcctcggcagacaagaccacggctactctacctctgtccttcaaggaggccacgctggctaagaggtttgctctcaagaccagaagtcagatcaccaagcggaagaggatgtcgctcatcaaggagaagaaggccgcccagacgctcagtgccatcttgctagccttcatcatcacgtggaccccctacaacatcatggtcctggtgaacaccttctgtgacagctgcatacccaaaacctattggaatctgggctactggctgtgctatatcaacagcaccgtgaaccctgtgtgctatgccctgtgcaacaaaacattcagaaccaccttcaagacgctcctcttgtgccagtgtgacaaaaggaagaggcgcaaacagcagtaccagcagagacagtcggtcatttttcacaagcgagtgccggaacaggccttg

SEQ ID NO: 10 - protein, 589 aa, Rattus norvegicus
MTLHSNSTTSPLFPNISSSWVHSPSEAGLPLGTVTQLGSYNISQETGNFSSNDTSSDPLGGHTIWQVVFIAFLTGFLALVTIIGNILVIVAFKVNKQLKTVNNYFLLSLACADLIIGVISMNLFTTYIIMNRWALGNLACDLWLSIDYVASNASVMNLLVISFDRYFSITRPLTYRAKRTTKRAGVMIGLAWVISFVLWAPAILFWQYFVGKRTVPPGECFIQFLSEPTITFGTAIAAFYMPVTIMTILYWRIYKETEKRTKELAGLQASGTEAEAENFVHPTGSSRSCSSYELQQQGVKRSSRRKYGRCHFWFTTKSWKPSAEQMDQDHSSSDSWNNNDAAASLENSASSDEEDIGSETRAIYSIVLKLPGHSSILNSTKLPSSDNLQVSNEDLGTVDVERNAHKLQAQKSMGDGDNCQKDFTKLPIQLESAVDTGKTSDTNSSADKTTATLPLSFKEATLAKRFALKTRSQITKRKRMSLIKEKKAAQTLSAILLAFIITWTPYNIMVLVNTFCDSCIPKTYWNLGYWLCYINSTVNPVCYALCNKTFRTTFKTLLLCQCDKRKRRKQQYQQRQSVIFHKRVPEQAL

SEQ ID NO: 11 - DNA, 1413 bp, synthetic construct
  source 1..1413: mol_type = other DNA
atgcgctgttctccaggcggcgtgtggctcgccctggctgcttcccttctgcacgttagcctgcagggtgagttccagcgcaaactgtataaggagcttgttaagaattataaccccctggagcggccggtcgcaaatgattcccagccactgacagtgtacttcagcctctccttgctgcagatcatggacgtggatgaaaagaaccaggtgctgaccactaatatttggttgcagatgtcctggaccgatcactacttgcagtggaatgtgagcgaatacccaggtgtaaagactgtaagattccctgacggccaaatctggaaaccagatatcctgctgtacaacagcgcagacgaaaggtttgatgcaacatttcacaccaacgtgggagtcaattcttcaggccactgcctgtacctgccccctggaatcttcaagtcctcatgctatatcgacgtccgctggtttcccttcgacgtccagcactgcaaactcaaattcgggagctggagctacggcggatggagcctggatctgcaaatgcaggaggctgacatctctggttacatcccgaatggggagtgggaccttgtgggaatccccggtaaaagaagcgagcgattttatgaatgctgcaaggaacccttccctgacgtaacattcacagttatcatcagaagaaggccattgttctacgccgttagtttgttgctccccagtatttttctcatggtcgtggacatcgtgggattttgtctcccacctgatagcggggagagggtctcctttaagattaccttgttgctcggctattctgtatttctgatcatcgtgtccgatacccttcctgccacaatcggcactccgctgataggagtgtatttcgtcgtgtgtatggcactcctggtgataagtctggcggaaactatcttcattgtacggctggtacataagcaggacctgcaaagacccgtgccagactggttgcgacaccttgtgctggacagaattgcatggattctgtgtcttggcgagcaacctatggcccaccggccacctgcaacctttcaagccaacaagacagacgattgtagtgggtctgatctgttgcctgctatggggaatcactgctcccatgttgggggaccacaagatttggaaaagaccccacgggggcggggatcaccccttcctcctccccgagaagcctctctcgctgtccgggggctgctccaggaactgtcaagcatccgacattttctggagaagcaggacgagatggacgaagtcgctgccgactggctgcgagtgggctacgtccttgacaggctgctgtttcggatctacttgctggcggtgctggcttattccattactctggtgacactctggtccatatggcactacagt

SEQ ID NO: 12 - protein, 471 aa, synthetic construct
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVGVNSSGHCLYLPPGIFKSSCYIDVRWFPFDVQHCKLKFGSWSYGGWSLDLQMQEADISGYIPNGEWDLVGIPGKRSERFYECCKEPFPDVTFTVIIRRRPLFYAVSLLLPSIFLMVVDIVGFCLPPDSGERVSFKITLLLGYSVFLIIVSDTLPATIGTPLIGVYFVVCMALLVISLAETIFIVRLVHKQDLQRPVPDWLRHLVLDRIAWILCLGEQPMAHRPPATFQANKTDDCSGSDLLPAMGNHCSHVGGPQDLEKTPRGRGSPLPPPREASLAVRGLLQELSSIRHFLEKQDEMDEVAADWLRVGYVLDRLLFRIYLLAVLAYSITLVTLWSIWHYS

SEQ ID NO: 13 - protein, 8 aa, synthetic construct
  VAR_SEQ 2: note = X is any amino acid
  VAR_SEQ 4: note = X is any amino acid
DXEXNPGP

SEQ ID NO: 14 - protein, 8 aa, synthetic construct
DVEENPGP

SEQ ID NO: 15 - protein, 461 aa, synthetic construct
MKSSAFSHPTYTLVWKVGILAVTLYYAIRIPLTLVFPSLFSPLLPLDILASLALIADIPLDFAFESRKTSGRKPTLLAPSRLPDLLAALPLDLLVFALHLPSPLSLLSLVRLLKLISVQRSATRILSYRINPALLRLLSLVGFILLAAHGIACGWMSLQPPSESPAGTRYLSAFYWTITTLTTIGYGDITPSTPIQTVYTIVIELLGAAMYGLVIGNIASLVSKLDAAKLLHRERMERVTAFLSYKKISPELQRRILEYFDYLWETRRGYEEREVLKELPHPLRLAVAMEIHGDVIEKVPLFKGAGEDFIRDIILHLEPVIYGPGEYIIRAGELGSDVYFINRGSVEVLSADEKTRYAILSEGQFFGEMALILRAPRTATVRARTFCDLYRLDKETFDRILSRYPEIAAQIQELAVRRKEELEGGTSRRGTGPGLKELACGSGATNFSLLKQAGDVEENPG

SEQ ID NO: 16 - protein, 413 aa, synthetic construct
PMTLHSNSTTSPLFPNISSSWVHSPSEAGLPLGTVTQLGSYNISQETGNFSSNDTSSDPLGGHTIWQVVFIAFLTGFLALVTIIGNILVIVAFKVNKQLKTVNNYFLLSLACADLIIGVISMNLFTTYIIMNRWALGNLACDLWLSIDCVASNASVMNLLVISFDRYFSITSPFRYQSLMTRARAGVMIGLAWVISFVLWAPAILFWQYFVGKRTVPPGECFIQFLSEPTITFGTAIAGFYMPVTIMTILYWRVYREAKEQIRKIDRCEGRFYGSQEQPQPPPLPQHQPILGNGRASKRKTSRVMAMREHKALQTLSAILLAFIITWTPYNIMVLVNTFCDSCIPKTYWNLGYWLCYINSTVNPVCYALCNKTFRTTFKTLLLCQCDKRKRRKQQYQQRQSVIFHKRVPEQAL

SEQ ID NO: 17 - DNA, 84 bp, synthetic construct
  source 1..84: mol_type = other DNA
ggacgtacggcggccaagagcaggatcaccagcgagggcgagtacatccccctggaccagatcgacatcaacgtggcggtaccc

SEQ ID NO: 18 - protein, 28 aa, synthetic construct
GRTAAKSRITSEGEYIPLDQIDINVAVP

SEQ ID NO: 19 - DNA, 711 bp, synthetic construct
  source 1..711: mol_type = other DNA
gtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgagctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgccatccacggcaacgtctatatcaccgccgacaagcagaagaacggcatcaaggccaacttcggcctcaactgcaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtat

SEQ ID NO: 20 - protein, 237 aa, synthetic construct
VSKGEELFTGVVPILVELDGDVNGHKFSVSGEGEGDATYGKLTLKFICTTGKLPVPWPTLVTTLSWGVQCFARYPDHMKQHDFFKSAMPEGYVQERTIFFKDDGNYKTRAEVKFEGDTLVNRIELKGIDFKEDGNILGHKLEYNAIHGNVYITADKQKNGIKANFGLNCNIEDGSVQLADHYQQNTPIGDGPVLLPDNHYLSTQSKLSKDPNEKRDHMVLLEFVTAAGITLGMDELY

SEQ ID NO: 21 - DNA, 309 bp, Rattus norvegicus
  source 1..309: mol_type = genomic DNA
gcagccctgcaggagaagaagtcatgcagccagcgcatggccgaattccggcaatactgttggaacccggacactgggcagatgctgggccgcaccccagcccggtgggtgtggatcagcctgtactatgcagctttctacgtggtcatgactgggctctttgccttgtgcatctatgtgctgatgcagaccattgatccctacacccccgactaccaggaccagttaaagtcaccgggggtaaccttgagaccggatgtgtatggggaaagagggctgcagatttcctacaacatctctgaaaacagc

SEQ ID NO: 22 - protein, 103 aa, Rattus norvegicus
AALQEKKSCSQRMAEFRQYCWNPDTGQMLGRTPARWVWISLYYAAFYVVMTGLFALCIYVLMQTIDPYTPDYQDQLKSPGVTLRPDVYGERGLQISYNISENS

SEQ ID NO: 23 - DNA, 45 bp, synthetic construct
  source 1..45: mol_type = other DNA
tctagacaggcccagatcaccggacgtccggagggagctgtacaa

SEQ ID NO: 24 - protein, 15 aa, synthetic construct
SRQAQITGRPEGAVQ

SEQ ID NO: 25 - DNA, 1155 bp, synthetic construct
  source 1..1155: mol_type = other DNA
ggacgtacggcggccaagagcaggatcaccagcgagggcgagtacatccccctggaccagatcgacatcaacgtggcggtacccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgagctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgccatccacggcaacgtctatatcaccgccgacaagcagaagaacggcatcaaggccaacttcggcctcaactgcaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtataccggtgcagccctgcaggagaagaagtcatgcagccagcgcatggccgaattccggcaatactgttggaacccggacactgggcagatgctgggccgcaccccagcccggtgggtgtggatcagcctgtactatgcagctttctacgtggtcatgactgggctctttgccttgtgcatctatgtgctgatgcagaccattgatccctacacccccgactaccaggaccagttaaagtcaccgggggtaaccttgagaccggatgtgtatggggaaagagggctgcagatttcctacaacatctctgaaaacagctctagacaggcccagatcaccggacgtccggagggagctgtacaa

SEQ ID NO: 26 - protein, 385 aa, synthetic construct
GRTAAKSRITSEGEYIPLDQIDINVAVPVSKGEELFTGVVPILVELDGDVNGHKFSVSGEGEGDATYGKLTLKFICTTGKLPVPWPTLVTTLSWGVQCFARYPDHMKQHDFFKSAMPEGYVQERTIFFKDDGNYKTRAEVKFEGDTLVNRIELKGIDFKEDGNILGHKLEYNAIHGNVYITADKQKNGIKANFGLNCNIEDGSVQLADHYQQNTPIGDGPVLLPDNHYLSTQSKLSKDPNEKRDHMVLLEFVTAAGITLGMDELYTGAALQEKKSCSQRMAEFRQYCWNPDTGQMLGRTPARWVWISLYYAAFYVVMTGLFALCIYVLMQTIDPYTPDYQDQLKSPGVTLRPDVYGERGLQISYNISENSSRQAQITGRPEGAVQ

SEQ ID NO: 27 - DNA, 3720 bp, synthetic construct
  source 1..3720: mol_type = other DNA
atgaaaagctccgccttctcccaccccacctacaccctggtctggaaagtcggcattctggctgtcactctgtattacgctattcgaatcccactgaccctggtgttcccctctctgtttagtcccctgctgcctctggatatcctggccagtctggctctgatcgcagacattcctctggatttcgcctttgagtcacgaaagacaagcggcaggaaaccaactctgctggctcctagccgactgccagatctgctggccgctctgccactggacctgctggtgttcgccctgcacctgccatcacccctgagcctgctgtccctggtgaggctgctgaagctgatctccgtccagaggtctgctacaagaatcctgtcttacagaattaacccagcactgctgcggctgctgagtctggtgggattcatcctgctggcagcccatgggattgcctgcggctggatgtcactgcagccacctagcgagtccccagcaggaaccagatacctgagcgccttctactggacaatcaccacactgactaccatcggctacggagatattaccccatccacacccattcagaccgtgtacaccatcgtcattgagctgctgggagctgcaatgtatggactggtcatcgggaatattgcatctctggtcagtaagctggacgccgctaaactgctgcaccgagagaggatggaacgggtgacagctttcctgagttacaagaaaatctcacctgagctgcagaggagaattctggaatactttgattatctgtgggagactcggcgcgggtatgaggaacgcgaggtgctgaaggaactgcctcacccactgcgactggctgtcgcaatggaaatccatggcgacgtgattgagaaggtcccactgttcaaaggggccggcgaagactttatccgcgatatcattctgcatctggagcccgtgatctacggacctggggaatatatcattagggctggcgagctgggcagcgatgtctactttatcaacagaggcagcgtggaggtcctgtccgcagacgaaaagacccggtatgccatcctgtctgagggccagttctttggagaaatggcactgattctgcgagcaccacgaacagctactgtgagagcacggactttctgtgacctgtacagactggataaagaaacctttgacagaatcctgtctcgctatcctgagattgcagcccagattcaggaactggctgtgcggaggaaagaagaactggaaggggggacatcacggcggggaaccggtcccgggcttaaggagctcgcatgcggaagcggaggacgtacggcggccaagagcaggatcaccagcgagggcgagtacatccccctggaccagatcgacatcaacgtggcggtacccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgagctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgccatccacggcaacgtctatatcaccgccgacaagcagaagaacggcatcaaggccaacttcggcctcaactgcaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtataccggtgcagccctgcaggagaagaagtcatgcagccagcgcatggccgaattccggcaatactgttggaacccggacactgggcagatgctgggccgcaccccagcccggtgggtgtggatcagcctgtactatgcagctttctacgtggtcatgactgggctctttgccttgtgcatctatgtgctgatgcagaccattgatccctacacccccgactaccaggaccagttaaagtcaccgggggtaaccttgagaccggatgtgtatggggaaagagggctgcagatttcctacaacatctctgaaaacagctctagacaggcccagatcaccggacgtccggagggagctgtacaaatgaccttgcacagtaacagtacaacctcgcctttgtttcccaacatcagctcttcctgggtgcacagtccctcggaggcagggctgcccttggggacagtcactcagttgggcagctacaacatttcacaagaaactgggaatttctcctcaaacgacacctccagcgaccctctcgggggtcacaccatctggcaagtggtcttcattgccttcttaactggcttcctggcattggtgaccatcattggcaacatccttgtcattgtggccttcaaggtcaacaaacagctgaagacagtcaacaactacttcctcttaagcctggcctgtgcagacctgatcatcggggtcatttccatgaacctgttcactacctacatcattatgaaccgttgggcactggggaacttagcctgcgacctctggctctccattgactgtgtggccagcaatgcctctgtcatgaatctgctggtcatcagctttgacaggtacttttccatcacttctccattccgctaccagagcctgatgaccagggctcgagctggtgtgatgattggtctggcttgggtcatctcctttgtcctatgggctcctgccatcttgttctggcaatactttgtagggaagagaactgtgcccccaggagaatgtttcattcagtttctgagtgagcccaccatcaccttcggcacggcgatcgctggcttttacatgcctgtcaccatcatgactattttatactggcgggtgtaccgggaggccaaggagcagatcaggaagatcgaccgctgcgagggccggttctatggcagccaggagcagccgcagccacccccgctcccccaacaccagcccatcctcggcaacggccgtgccagcaagaggaagacgtcccgtgtcatggccatgagggaacacaaagctctgcagacgctcagtgccatcttgctggccttcatcatcacgtggaccccctacaacatcatggtcctggtgaacaccttctgtgacagctgcatacccaaaacctattggaatctgggctactggctgtgctatatcaacagcaccgtgaaccctgtgtgctatgccctgtgcaacaaaacattcagaaccaccttcaagacgctcctcttgtgccagtgtgacaaaaggaagaggcgcaaacagcagtaccagcagagacagtcggtcatttttcacaagcgagtgccggagcaggccttg

SEQ ID NO: 28 - protein, 1240 aa, synthetic construct
MKSSAFSHPTYTLVWKVGILAVTLYYAIRIPLTLVFPSLFSPLLPLDILASLALIADIPLDFAFESRKTSGRKPTLLAPSRLPDLLAALPLDLLVFALHLPSPLSLLSLVRLLKLISVQRSATRILSYRINPALLRLLSLVGFILLAAHGIACGWMSLQPPSESPAGTRYLSAFYWTITTLTTIGYGDITPSTPIQTVYTIVIELLGAAMYGLVIGNIASLVSKLDAAKLLHRERMERVTAFLSYKKISPELQRRILEYFDYLWETRRGYEEREVLKELPHPLRLAVAMEIHGDVIEKVPLFKGAGEDFIRDIILHLEPVIYGPGEYIIRAGELGSDVYFINRGSVEVLSADEKTRYAILSEGQFFGEMALILRAPRTATVRARTFCDLYRLDKETFDRILSRYPEIAAQIQELAVRRKEELEGGTSRRGTGPGLKELACGSGGRTAAKSRITSEGEYIPLDQIDINVAVPVSKGEELFTGVVPILVELDGDVNGHKFSVSGEGEGDATYGKLTLKFICTTGKLPVPWPTLVTTLSWGVQCFARYPDHMKQHDFFKSAMPEGYVQERTIFFKDDGNYKTRAEVKFEGDTLVNRIELKGIDFKEDGNILGHKLEYNAIHGNVYITADKQKNGIKANFGLNCNIEDGSVQLADHYQQNTPIGDGPVLLPDNHYLSTQSKLSKDPNEKRDHMVLLEFVTAAGITLGMDELYTGAALQEKKSCSQRMAEFRQYCWNPDTGQMLGRTPARWVWISLYYAAFYVVMTGLFALCIYVLMQTIDPYTPDYQDQLKSPGVTLRPDVYGERGLQISYNISENSSRQAQITGRPEGAVQMTLHSNSTTSPLFPNISSSWVHSPSEAGLPLGTVTQLGSYNISQETGNFSSNDTSSDPLGGHTIWQVVFIAFLTGFLALVTIIGNILVIVAFKVNKQLKTVNNYFLLSLACADLIIGVISMNLFTTYIIMNRWALGNLACDLWLSIDCVASNASVMNLLVISFDRYFSITSPFRYQSLMTRARAGVMIGLAWVISFVLWAPAILFWQYFVGKRTVPPGECFIQFLSEPTITFGTAIAGFYMPVTIMTILYWRVYREAKEQIRKIDRCEGRFYGSQEQPQPPPLPQHQPILGNGRASKRKTSRVMAMREHKALQTLSAILLAFIITWTPYNIMVLVNTFCDSCIPKTYWNLGYWLCYINSTVNPVCYALCNKTFRTTFKTLLLCQCDKRKRRKQQYQQRQSVIFHKRVPEQAL